(19) United States
(12) Patent Application Publication
(10) Pub. No.: US 2012/0058154 A1
(43) Pub. Date: Mar. 8, 2012

(54) SYNTHETIC NANOCARRIER VACCINES COMPRISING PEPTIDES OBTAINED OR DERIVED FROM HUMAN INFLUENZA A VIRUS M2E

(75) Inventors: Petr Ilyinskii, Cambridge, MA (US); Yun Gao, Southborough, MA (US); Grayson B. Lipford, Watertown, MA (US)

(73) Assignee: Selecta Biosciences, Inc., Watertown, MA (US)

(21) Appl. No.: 13/213,552

(22) Filed: Aug. 19, 2011

Related U.S. Application Data
(60) Provisional application No. 61/375,543, filed on Aug. 20, 2010.
(60) Provisional application No. 61/375,635, filed on Aug. 20, 2010.
(60) Provisional application No. 61/375,586, filed on Aug. 20, 2010.

(51) Int. Cl. A61K 39/385; C07K 17/02; C07K 17/14; C07K 17/08; A61K 39/145; A61P 37/04; A61P 31/16; C08G 63/91; C07K 17/00; C07K 14/11; B82Y 5/00

(52) U.S. Cl. 424/400; 530/345; 530/325; 530/326; 530/324; 424/196.11; 530/300; 424/209.1; 525/54.1; 977/917

(57) ABSTRACT

This invention relates to compositions and methods that can be used immunize a subject against influenza. Generally, the compositions and methods include peptides obtained or derived from human influenza A virus M2 protein.

### RELATED APPLICATIONS

This application claims the benefit under 35 U.S.C. §119 of U.S. provisional applications 61/375,586, 61/375,635, and 61/375,543, each filed Aug. 20, 2010, the entire contents of each of which are incorporated herein by reference.

### FIELD OF THE INVENTION

This invention relates to compositions and methods that can be used immunize a subject against influenza. Generally, the compositions and methods include peptides obtained or derived from human influenza A virus M2E.

### BACKGROUND OF THE INVENTION

Influenza is an infectious disease caused by RNA viruses of the family Orthomyxoviridae. Common symptoms of the disease include chills, fever, sore throat, muscle pains, severe headache, coughing, and fatigue. In more serious cases, influenza can lead to pneumonia, which can be fatal. Influenza spreads around the world in seasonal epidemics, resulting in the deaths of between 250,000 and 500,000 people every year, and up to millions in some pandemic years. Human influenza A virus (“HIAV”) is the most common strain of the virus, and is responsible for all major influenza pandemics.

### SUMMARY OF THE INVENTION

In one aspect, a dosage form comprising synthetic nanocarriers coupled to peptides that are obtained or derived from an ectodomain region of human influenza A virus M2 protein is provided. In one embodiment, the dosage form further comprises a pharmaceutically acceptable excipient. In another embodiment, the peptides comprise a peptide obtained or derived from a peptide with an amino acid sequence as set forth in any of SEQ ID NOs: 1-17, 19-21, 23, 25 and 26 or any of the sequences provided in FIG. 5. In some embodiments, the peptides coupled to the synthetic nanocarriers are of the same type (i.e., are identical). In other embodiments, two or more types of peptides are coupled to the synthetic nanocarriers.

In still another embodiment, the synthetic nanocarriers are further coupled to one or more adjuvants. In one embodiment, the one or more adjuvants comprise Pluronic® block co-polymers, specifically modified or prepared peptides, stimulators or agonists of pattern recognition receptors, mineral salts, alum, alum combined with monophosphoryl lipid (MPL) A of Enterobacteria, MPL® (AS04), saponins, QS-21, Quil-A, ISCOMs, ISCOMATRIX™, MF59™, Montanide® ISA 51, Montanide® ISA 720, AS02, liposomes and liposomal formulations, AS01, synthesized or specifically prepared microparticles and microcarriers, bacteria-derived outer membrane vesicles of N. gonorrheae or Chlamydia trachomatis, chitosan particles, depot-forming agents, muramyl dipeptide, aminoalkyl glucosaminide 4-phosphates, RC529, bacterial toxoids, toxin fragments, agonists of Toll-Like Receptors 2, 3, 4, 5, 7, 8, 9 and/or combinations thereof; adenine derivatives; immunostimulatory DNA; immunostimulatory RNA; imidazoquinoline amines, imidazopyridine amines, 6,7-fused cycloalkylimidazopyridine amines, 1,2-bridged imidazoquinoline amines; imiquimod; resiquimod; type I interferons; bacterial lipopolysacccharide (LPS); VSV-G; HMGB-1; flagellin or portions or derivatives thereof; or immunostimulatory DNA molecules comprising CpGs, agonists for DC surface molecule CD40; type I interferons; poly I:C; poly I:C12U; bacterial lipopolysacccharide (LPS); VSV-G; HMGB-1; flagellin or portions or derivatives thereof; immunostimulatory DNA molecules comprising CpGs; proinflammatory stimuli released from necrotic cells; urate crystals; activated components of the complement cascade; activated components of immune complexes; complement receptor agonists; cytokines; or cytokine receptor agonists. In another embodiment, the one or more adjuvants comprise agonists of Toll-Like Receptors 2, 3, 4, 5, 7, 8, 9 and/or combinations thereof; adenine derivatives; immunostimulatory DNA; immunostimulatory RNA; imidazoquinoline amines, imidazopyridine amines, 6,7-fused cycloalkylimidazopyridine amines, 1,2-bridged imidazoquinoline amines, imiquimod, resiquimod, immunostimulatory DNA molecules comprising CpGs, poly I:C or poly I:C12U.

In one embodiment, the synthetic nanocarriers comprise lipid-based nanoparticles, polymeric nanoparticles, metallic nanoparticles, surfactant-based emulsions, dendrimers, buckyballs, nanowires, virus-like particles, peptide or protein-based particles, lipid-polymer nanoparticles, spheroidal nanoparticles, cubic nanoparticles, pyramidal nanoparticles, oblong nanoparticles, cylindrical nanoparticles, or toroidal nanoparticles. In another embodiment, the synthetic nanocarriers comprise poly(lactic acid)-polyethyleneglycol copolymer, poly(glycolic acid)-polyethyleneglycol copolymer, or poly(lactic-co-glycolic acid)-polyethyleneglycol copolymer.

In still another embodiment, the synthetic nanocarriers comprise a T-helper antigen. In one embodiment, the T-helper antigen is coupled to the synthetic nanocarriers. In another embodiment, the T-helper antigen is any of the T-helper antigens provided herein. In still another embodiment, the amino acid sequence of the T-helper antigen comprises the amino acid sequence as set forth in SEQ ID NO: 18 or 22.

In a further embodiment, the synthetic nanocarriers are present in an amount effective to provide an immune response to the peptides when administered to a subject.

In still a further embodiment, the dosage form further comprises influenza antigen that is not coupled to the synthetic nanocarriers.

In one embodiment, at least a portion of the peptides that are obtained or derived from an ectodomain region of human influenza A virus M2 protein are coupled to a surface of the synthetic nanocarriers. In another embodiment, the synthetic nanocarriers are covalently coupled to peptides that are obtained or derived from an ectodomain region of human influenza A virus M2 protein. In still another embodiment, the synthetic nanocarriers are non-covalently coupled to peptides that are obtained or derived from an ectodomain region of human influenza A virus M2 protein.

In another embodiment, the dosage form or the synthetic nanocarriers comprised therein generate(s) in a subject polyclonal antibodies that compete for binding to human influenza A virus M2 protein with a control antibody, wherein the control antibody is 14C2.

In another aspect, a dosage form comprising peptides that are obtained or derived from an ectodomain region of human influenza A virus M2 protein that generates in a subject polyclonal antibodies that compete for binding to human influenza A virus M2 protein with a control antibody, wherein the control antibody is 14C2, is provided.

In one embodiment, whether or not the polyclonal antibodies compete for binding is assessed with any of the methods described herein. In another embodiment, the competitive binding is assessed using the entire human influenza A virus M2 protein. In yet another embodiment, the competitive binding is assessed using the ectodomain region of human influenza A virus M2 protein. In still another embodiment, the competitive binding is assessed using a peptide with an amino acid sequence as set forth in any one of SEQ ID NOs: 1-17, 19-21, 23, 25 and 26 or any of the sequences provided in FIG. 5. In still another embodiment, the competitive binding is assessed using any of the peptides provided herein.

In one embodiment, the dosage form further comprises a pharmaceutically acceptable excipient. In another embodiment, the peptides comprise a peptide obtained or derived from a peptide with an amino acid sequence as set forth in any of SEQ ID NOs: 1-17, 19-21, 23, 25 and 26 or any of the sequences provided in FIG. 5. In some embodiments, the peptides of the dosage form are of the same type (i.e., are identical). In other embodiments, the peptides comprise peptides of two or more types.

In yet another embodiment, the dosage form further comprises one or more adjuvants. In one embodiment, the one or more adjuvants comprise Pluronic® block co-polymers, specifically modified or prepared peptides, stimulators or agonists of pattern recognition receptors, mineral salts, alum, alum combined with monphosphoryl lipid (MPL) A of Enterobacteria, MPL® (AS04), saponins, QS-21, Quil-A, ISCOMs, ISCOMATRIX™, MF59™, Montanide® ISA 51, Montanide® ISA 720, AS02, liposomes and liposomal formulations, AS01, synthesized or specifically prepared microparticles and microcarriers, bacteria-derived outer membrane vesicles of N. gonorrheae or Chlamydia trachomatis, chitosan particles, depot-forming agents, muramyl dipeptide, aminoalkyl glucosaminide 4-phosphates, RC529, bacterial toxoids, toxin fragments, agonists of Toll-Like Receptors 2, 3, 4, 5, 7, 8, 9 and/or combinations thereof; adenine derivatives; immunostimulatory DNA; immunostimulatory RNA; imidazoquinoline amines, imidazopyridine amines, 6,7-fused cycloalkylimidazopyridine amines, 1,2-bridged imidazoquinoline amines; imiquimod; resiquimod; type I interferons; bacterial lipopolysacccharide (LPS); VSV-G; HMGB-1; flagellin or portions or derivatives thereof; or immunostimulatory DNA molecules comprising CpGs, agonists for DC surface molecule CD40; type I interferons; poly I:C; poly I:C12U; bacterial lipopolysacccharide (LPS); VSV-G; HMGB-1; flagellin or portions or derivatives thereof; immunostimulatory DNA molecules comprising CpGs; proinflammatory stimuli released from necrotic cells; urate crystals; activated components of the complement cascade; activated components of immune complexes; complement receptor agonists; cytokines; or cytokine receptor agonists. In another embodiment, the one or more adjuvants comprise agonists of Toll-Like Receptors 2, 3, 4, 5, 7, 8, 9 and/or combinations thereof; adenine derivatives; immunostimulatory DNA; immunostimulatory RNA; imidazoquinoline amines, imidazopyridine amines, 6,7-fused cycloalkylimidazopyridine amines, 1,2-bridged imidazoquinoline amines, imiquimod, resiquimod, immunostimulatory DNA molecules comprising CpGs, poly I:C or poly I:C12U.

In yet another embodiment, the dosage form further comprises T-helper antigens.

In still another embodiment, the dosage form further comprises a carrier that boosts an immune response to the peptides when the dosage form or peptides is/are administered to a subject. In one embodiment, the peptides are coupled to the carrier. In a further embodiment, a linking group couples the peptides to the carrier. In another embodiment, the T-helper antigens and/or one or more adjuvants are also coupled to the carrier. In one embodiment, the carrier comprises keyhole limpet hemocyanin, concholepas concholepas hemocyanin, bovine serum albumin, cationized BSA or ovalbumin. In another embodiment, the carrier comprises a synthetic nanocarrier.

In one embodiment, the synthetic nanocarrier comprises a/an lipid-based nanoparticle, polymeric nanoparticle, metallic nanoparticle, surfactant-based emulsion, dendrimer, buckyball, nanowire, virus-like particle, peptide or protein-based particle, lipid-polymer nanoparticle, spheroidal nanoparticle, cubic nanoparticle, pyramidal nanoparticle, oblong nanoparticle, cylindrical nanoparticle, or toroidal nanoparticle. In another embodiment, the synthetic nanocarrier comprises poly(lactic acid)-polyethyleneglycol copolymer, poly(glycolic acid)-polyethyleneglycol copolymer, or poly(lactic-co-glycolic acid)-polyethyleneglycol copolymer.

In one embodiment, the dosage form or peptides is/are in an amount effective to provide an immune response to the peptides when administered to a subject.

In another embodiment, the dosage form further comprises influenza antigen. In one embodiment, when the dosage form comprises a carrier, the influenza antigen is not coupled to the carrier. In another embodiment, the carrier is a synthetic nanocarrier.

In another aspect, a method comprising administering any of the dosage forms provided to a subject is provided. In one embodiment, the dosage form is administered at least once to the subject. In another embodiment, the dosage form is administered at least twice to the subject. In still another embodiment, the dosage form is administered at least three times to the subject. In yet another embodiment, the dosage form is administered at least four times to the subject.

In yet another aspect, a method comprising providing synthetic nanocarriers, and coupling peptides that are obtained or derived from an ectodomain region of human influenza A virus M2 protein to the synthetic nanocarriers is provided. In one embodiment, the coupling comprises covalently coupling the peptides to the synthetic nanocarriers.

In yet another aspect, a composition, dosage form or vaccine obtained, or obtainable, by any of the methods provided is provided.

In still another aspect, a process for producing a composition, dosage form or vaccine comprising the steps of providing synthetic nanocarriers, and coupling peptides that are obtained or derived from an ectodomain region of human influenza A virus M2 protein to the synthetic nanocarriers is provided.

In a further aspect, any of the dosage forms provided may be for use in therapy or prophylaxis.

In still a further aspect, any of the dosage forms provided may be for use in any of the methods provided herein.

In yet a further aspect, any of the dosage forms provided may be for use in vaccination.

In another aspect, any of the dosage forms provided may be for use in a method of therapy or prophylaxis of influenza virus infection, for example influenza A virus infection.

In yet another aspect, any of the dosage forms provided may be for use in a method of therapy or prophylaxis comprising administration by a subcutaneous, intramuscular, intradermal, oral, intranasal, transmucosal, sublingual, rectal; ophthalmic, transdermal, transcutaneous route or by a combination of these routes.

In still another aspect, a use of any of the dosage forms provided may be for the manufacture of a medicament, for example a vaccine, for use in any of the methods provided.

### BRIEF DESCRIPTION OF THE DRAWINGS

FIG. 1 shows anti-M2e antibody titers after NC-M2e vaccination. Groups 1 and 3 were immunized with NC-M2e (R848+ ovalbumin memory peptide); groups 2 and 4 were immunized with NC-M2e (R848, no ovalbumin memory peptide); group 5 was immunized with M2e peptide only; group 6 was immunized with M2e peptide (20 μg) with alum adjuvant.

FIG. 2 demonstrates that NC-M2e express M2e peptide that is recognized by a monoclonal anti-M2 protein antibody.

FIG. 3 shows anti-M2e antibody titers from M2e peptide vaccination using NC-M2e (C6 PEG) or NC-M2e (PEG3 PEG).

FIG. 4 shows titers from M2e peptide vaccination using NC-M2e in the presence of nanocarriers containing other proteins or peptides.

FIG. 5 provides further exemplary peptides from which the peptides of the compositions and methods provided can be obtained or derived. The described sequences correspond, from top to bottom, to SEQ ID NOs 1-13.

### DETAILED DESCRIPTION OF THE INVENTION

### I. Introduction

While protective immunity against HIAV can be attained by many currently available vaccine methodologies and approaches, none of them provides for a long-term and/or broad cross-strain protection. Due to perpetual changes in structure of two major HIAV surface proteins, hemagglutinin (HA) and neuraminidase (NA), annual re-vaccinations of general populations are necessary. Moreover, in the event of pandemic caused by a dramatically novel influenza strain (such as the so-called “swine” H1N1 influenza of 2009-2010), targeted vaccination against this newly-derived strain is needed. While both re-vaccination and manufacturing of efficient vaccines against novel HIAV strains are possible and attainable, both of them necessitate repeated expenditure of resources, suffer from decreased effectiveness due to possible mismatches between vaccinating and pathogenic strains, and require a significant time lag between initiation of vaccine manufacturing and its availability to general public. All of the above necessitates continuous and significant investment of public and medical efforts targeting HIAV, encompassing constant epidemiologic surveillance of influenza and annual re-vaccination of susceptible populations with vaccines. Therefore, what is needed are compositions and methods that could address the problems noted above that are associated with producing vaccines against human influenza A virus.

The inventors have unexpectedly and surprisingly discovered that the problems and limitations noted above can be overcome by practicing the invention disclosed herein. In particular, the inventors have unexpectedly discovered that it is possible to provide dosage forms, and related methods comprising: synthetic nanocarriers coupled to peptides that are obtained or derived from an ectodomain region of human influenza A virus M2 protein. In another embodiment, the inventors have unexpectedly discovered that it is possible to provide methods that comprise providing synthetic nanocarriers; and coupling peptides that are obtained or derived from an ectodomain region of human influenza A virus M2 protein to the synthetic nanocarriers.

An approach aiming to circumvent ever-occurring antigenic changes in HA is to create a vaccine capable of inducing protective immune response directed against another viral surface protein, M2. M2 of HIAV is instrumental for viral uncoating inside the cell and, thus, for virus infectivity. Moreover, M2 is especially abundant on surface of virus-infected cells where it forms ion channels essential for viral replication. Differently from HA, M2 (and particularly its external region) is highly conserved among widely divergent viral strains. This external region termed ectodomain (M2e) comprises only 24 amino acids. It is known that immunity against M2e is broadly protective against influenza infection (Mozdzanowska et al., 2003; Wang et al., 2008), with M2e-binding antibodies being capable of preventing and/or alleviating the virus-induced disease (Neirynck et al., 1999; Treanor et al., 1990; Wang et al., 2008; Beerli et al., 2009).

At the same time, the generation of robust antibody response to M2e (whether in form of a separate peptide or as a part of full-size M2 protein) is notoriously difficult (Slepushkin et al., 1995; Jegerlehner et al, 2004; De Filette et al., 2006a). Therefore, many different modalities such as carrier proteins (e.g., hepatitis virus surface antigen, or keyhole limpet hemocyanin), novel adjuvants (e.g., bacterial flagellin), vectors (DNA), carriers, and vaccination schemes have been utilized to augment humoral immune response to influenza M2 and M2e with various degrees of success (Black et al., 1993; Fan et al., 2004; Ernst et al., 2006; Denis et al., 2008; Huleatt et al., 2008; Tompkins et al., 2007; Mozdzanowska et el., 2007; De Filette et al., 2006ab; 2008ab; Jimenez et al., 2007; reviewed in Schotsaert et al., 2009). Although sufficient protection has been demonstrated in a number of research settings, all of the above-mentioned approaches suffered from different drawbacks such as an induction of potentially dangerous inflammatory side-effects (Huleatt et. al., 2008), necessity to use an exceedingly high vaccination dose (Tompkins et al., 2007), or to employ the fusion to other immunogenic (and potentially, allergy-inducing) proteins (De Filette et al., 2008b).

The present invention addresses the problems found in the art by providing a viral antigenic conserved M2e peptide (comprising, in an embodiment, amino acid residues 1-21) coupled to synthetic nanocarriers. Further modifications of M2e may comprise addition of two C-terminal glycines with acetylene group at C-terminus to enable efficient coupling of peptide to synthetic nanocarriers while maintaining its natural conformation. Additionally, in embodiments, the synthetic nanocarriers may comprise an adjuvant such as the TLR7/8 agonist R848 and a T-cell helper antigen.

Examples 4 and 5 provide experimental evidence illustrating that, in some embodiments, immunization with novel M2e-synthetic nanocarriers (M2e-NC) resulted in generation of a highly potent M2e-carrying immunogen capable of efficiently inducing anti-M2 antibody response in vivo. Notably, synthetic nanocarrier-based M2e immunogens according to the invention possess relatively few undesirable features of conventional M2e-based vaccines while providing for a robust and potentially cross-protective anti-M2 antibody response. Accordingly, dosage forms according to the present invention potentially provide cross-protective immunity against variable strains of HIAV. Moreover, the inventive dosage forms are completely synthetic and thus especially safe and easy to manufacture.

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

All publications, patents and patent applications cited herein, whether supra or infra, are hereby incorporated by reference in their entirety for all purposes.

As used in this specification and the appended claims, the singular forms “a,” “an” and “the” include plural referents unless the content clearly dictates otherwise. For example, reference to “a polymer” includes a mixture of two or more such molecules or a mixture of differing molecular weights of a single polymer species, reference to “a synthetic nanocarrier” includes a mixture of two or more such synthetic nanocarriers or a plurality of such synthetic nanocarriers, reference to a “DNA molecule” includes a mixture of two or more such DNA molecules or a plurality of such DNA molecules, reference to “an adjuvant” includes a mixture of two or more such materials or a plurality of adjuvant molecules, and the like.

As used herein, the term “comprise” or variations thereof such as “comprises” or “comprising” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein, the term “comprising” is inclusive and does not exclude additional, unrecited integers or method/process steps.

In embodiments of any of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, element, characteristics, properties, method/process steps or limitations) alone.

The invention will be described in more detail below.

II. Definitions “14C2” is anti-influenza A M2 monoclonal antibody available, for example, from Thermo Scientific, catalog #MA1-082 and described in U.S. Application Publication No. 2009/0162400 A1.

“Adjuvant” means an agent that does not constitute a specific antigen, but boosts the strength and longevity of immune response to a concomitantly administered antigen. Such adjuvants may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherihia coli, Salmonella minnesota, Salmonella typhimurium, or Shigella flexneri or specifically with MPL® (AS04), MPL A of above-mentioned bacteria separately, saponins, such as QS-21, Quil-A, ISCOMs, ISCOMATRIX™, emulsions such as MF59™ Montanide® ISA 51 and ISA 720, AS02 (QS21+squalene+MPL®), liposomes and liposomal formulations such as AS01, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic® block co-polymers, specifically modified or prepared peptides, such as muramyl dipeptide, aminoalkyl glucosaminide 4-phosphates, such as RC529, or proteins, such as bacterial toxoids or toxin fragments.

In some embodiments, adjuvants comprise agonists for pattern recognition receptors (PRR), including, but not limited to Toll-Like Receptors (TLRs), specifically TLRs 2, 3, 4, 5, 7, 8, 9 and/or combinations thereof. In other embodiments, adjuvants comprise agonists for Toll-Like Receptors 3, agonists for Toll-Like Receptors 7 and 8, or agonists for Toll-Like Receptor 9; preferably the recited adjuvants comprise imidazoquinolines; such as R848 (also known as resiquimod); adenine derivatives, such as those disclosed in U.S. Pat. No. 6,329,381 (Sumitomo Pharmaceutical Company); US Published Patent Application 2010/0075995 to Biggadike et al., or WO 2010/018132 to Campos et al.; immunostimulatory DNA; or immunostimulatory RNA.

In specific embodiments, synthetic nanocarriers incorporate as adjuvants compounds that are agonists for toll-like receptors (TLRs) 7 & 8 (“TLR 7/8 agonists”). Of utility are the TLR 7/8 agonist compounds disclosed in U.S. Pat. No. 6,696,076 to Tomai et al., including but not limited to imidazoquinoline amines, imidazopyridine amines, 6,7-fused cycloalkylimidazopyridine amines, and 1,2-bridged imidazoquinoline amines. Preferred adjuvants comprise imiquimod and resiquimod (R848). In specific embodiments, an adjuvant may be an agonist for the DC surface molecule CD40. In certain embodiments, to stimulate immunity rather than tolerance, a synthetic nanocarrier incorporates an adjuvant that promotes DC maturation (needed for priming of naive T cells) and the production of cytokines, such as type I interferons, which promote antibody immune responses.

In embodiments, adjuvants also may comprise immunostimulatory RNA molecules, such as but not limited to dsRNA, poly I:C or poly I:poly C12U (available as Ampligen®, both poly I:C and poly I:poly C12U being known as TLR3 stimulants), and/or those disclosed in F. Heil et al., “Species-Specific Recognition of Single-Stranded RNA via Toll-like Receptor 7 and 8” Science 303(5663), 1526-1529 (2004); J. Vollmer et al., “Immune modulation by chemically modified ribonucleosides and oligoribonucleotides” WO 2008033432 A2; A. Forsbach et al., “Immunostimulatory oligoribonucleotides containing specific sequence motif(s) and targeting the Toll-like receptor 8 pathway” WO 2007062107 A2; E. Uhlmann et al., “Modified oligoribonucleotide analogs with enhanced immunostimulatory activity” U.S. Pat. Appl. Publ. US 2006241076; G. Lipford et al., “Immunostimulatory viral RNA oligonucleotides and use for treating cancer and infections” WO 2005097993 A2; G. Lipford et al., “Immunostimulatory G,U-containing oligoribonucleotides, compositions, and screening methods” WO 2003086280 A2. In some embodiments, an adjuvant may be a TLR-4 agonist, such as bacterial lipopolysacccharide (LPS), VSV-G, and/or HMGB-1. In some embodiments, adjuvants may comprise TLR-5 agonists, such as flagellin, or portions or derivatives thereof, including but not limited to those disclosed in U.S. Pat. Nos. 6,130,082, 6,585,980, and 7,192,725.

In specific embodiments, synthetic nanocarriers incorporate a ligand for Toll-like receptor (TLR)-9, such as immunostimulatory DNA molecules comprising CpGs, which induce type I interferon secretion, and stimulate T and B cell activation leading to increased antibody production and cytotoxic T cell responses (Krieg et al., CpG motifs in bacterial DNA trigger direct B cell activation. Nature. 1995. 374:546-549; Chu et al. CpG oligodeoxynucleotides act as adjuvants that switch on T helper 1 (Th1) immunity. J. Exp. Med. 1997. 186:1623-1631; Lipford et al. CpG-containing synthetic oligonucleotides promote B and cytotoxic T cell responses to protein antigen: a new class of vaccine adjuvants. Eur. J. Immunol. 1997. 27:2340-2344; Roman et al. Immunostimulatory DNA sequences function as T helper-1-promoting adjuvants. Nat. Med. 1997. 3:849-854; Davis et al. CpG DNA is a potent enhancer of specific immunity in mice immunized with recombinant hepatitis B surface antigen. J. Immunol. 1998. 160:870-876; Lipford et al., Bacterial DNA as immune cell activator. Trends Microbiol. 1998. 6:496-500; U.S. Pat. No. 6,207,646 to Krieg et al.; U.S. Pat. No. 7,223,398 to Tuck et al.; U.S. Pat. No. 7,250,403 to Van Nest et al.; or U.S. Pat. No. 7,566,703 to Krieg et al.

In some embodiments, adjuvants may be proinflammatory stimuli released from necrotic cells (e.g., urate crystals). In some embodiments, adjuvants may be activated components of the complement cascade (e.g., CD21, CD35, etc.). In some embodiments, adjuvants may be activated components of immune complexes. The adjuvants also include complement receptor agonists, such as a molecule that binds to CD21 or CD35. In some embodiments, the complement receptor agonist induces endogenous complement opsonization of the synthetic nanocarrier. In some embodiments, adjuvants are cytokines, which are small proteins or biological factors (in the range of 5 kD-20 kD) that are released by cells and have specific effects on cell-cell interaction, communication and behavior of other cells. In some embodiments, the cytokine receptor agonist is a small molecule, antibody, fusion protein, or aptamer.

In embodiments, at least a portion of the dose of adjuvant may be coupled to synthetic nanocarriers, preferably, all of the dose of adjuvant is coupled to synthetic nanocarriers. In other embodiments, at least a portion of the dose of the adjuvant is not coupled to the synthetic nanocarriers. In embodiments, the dose of adjuvant comprises two or more types of adjuvants. For instance, and without limitation, adjuvants that act on different TLR receptors may be combined. As an example, in an embodiment a TLR 7/8 agonist may be combined with a TLR 9 agonist. In another embodiment, a TLR 7/8 agonist may be combined with a TLR 4 agonist. In yet another embodiment, a TLR 9 agonist may be combined with a TLR 3 agonist.

“Administering” or “administration” means providing a drug to a subject in a manner that is pharmacologically useful.

“Amount effective” is any amount of a composition provided herein that produces one or more desired immune responses. This amount can be for in vitro or in vivo purposes. For in vivo purposes, the amount can be one that a clinician would believe may have a clinical benefit for a subject at risk of contracting an influenza infection, e.g., a human influenza A virus infection. Amounts effective include amounts that generate a humoral and/or cytotoxic T lymphocyte immune response, or certain levels thereof. An amount that is effective to produce a desired immune responses as provided herein can also be an amount that produces a desired therapeutic endpoint or a desired therapeutic result (e.g., prevents or reduces the severity of influenza infection in a subject).

A subject's immune response can be monitored by routine methods. Amounts effective will depend, of course, on the particular subject being treated; the severity of a condition, disease or disorder; the individual patient parameters including age, physical condition, size and weight; the duration of the treatment; the nature of concurrent therapy (if any); the specific route of administration and like factors within the knowledge and expertise of the health practitioner. These factors are well known to those of ordinary skill in the art and can be addressed with no more than routine experimentation. It is generally preferred that a maximum dose be used, that is, the highest safe dose according to sound medical judgment. It will be understood by those of ordinary skill in the art, however, that a patient may insist upon a lower dose or tolerable dose for medical reasons, psychological reasons or for virtually any other reasons.

“Antigen” means a B cell antigen or T cell antigen. In embodiments, antigens are coupled to the synthetic nanocarriers. In other embodiments, antigens are not coupled to the synthetic nanocarriers. In embodiments antigens are coadministered with the synthetic nanocarriers. In other embodiments antigens are not coadministered with the synthetic nanocarriers. “Type(s) of antigens” means molecules that share the same, or substantially the same, antigenic characteristics.

“At least a portion of the dose” means at least some part of the dose, ranging up to including all of the dose.

“B cell antigen” means any antigen that is or recognized by and triggers an immune response in a B cell (e.g., an antigen that is specifically recognized by a B cell receptor on a B cell). In some embodiments, an antigen that is a T cell antigen is also a B cell antigen. In other embodiments, the T cell antigen is not also a B cell antigen.

“Competes for binding” refers to any competitive inhibition of binding to a target antigen (e.g., inhibition of the binding of a control antibody to a target antigen by polyclonal antibodies generated from the methods or compositions provided herein). Such inhibition can be identified in a simple immunoassay showing the ability of the polyclonal antibodies to block the binding of the control antibody to a target antigen. In some embodiments of such assays, the target antigen is the entire human influenza A virus M2 protein. In other embodiments, the target antigen is the ectodomain region of human influenza A virus M2 protein. In another embodiment, the target antigen is a peptide comprising an amino acid sequence as set forth in any one of SEQ ID NOs: 1-17, 19-21, 23, 25 and 26 or any of the sequences provided in FIG. 5. In still another embodiment, the target antigen is any of the peptides provided herein.

Competitive binding is found when the binding of the polyclonal antibodies that are generated with the compositions or methods provided herein inhibit the specific binding of the control antibody, 14C2, to a target antigen, examples of which are provided above. In some embodiments, the polyclonal antibodies inhibit the specific binding of the control antibody by at least 5%, 10%, 15%, 20%, 25%, 30%, 35%, 40%, 45%, 50%, 55%, 60%, 65%, 70%, 75%, 80%, 85%, 90%, 95% or more. Numerous types of competitive binding assays are known, for example: solid phase direct or indirect radioimmunoassay (RIA); solid phase direct or indirect enzyme immunoassay (EIA) sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)).

As an example, such an assay may involve the use of target antigen bound to a solid surface, unlabeled polyclonal antibodies to be tested (in excess) and a labeled control antibody. Competitive inhibition may be measured by determining the amount of label bound to the solid surface in the presence of the control antibody. In an embodiment of such an assay, the polyclonal antibodies are incubated with target antigen bound to a solid surface in the presence of biotinylated control antibody. Following washing, horseradish peroxidase (HRP)-conjugated streptavidin is added to determine the amount of bound biotinylated control antibody. After addition of a substrate (TMB), the development of the enzymatic reaction is stopped with sulfuric acid and absorbance is measured. The percent inhibition is defined relative to the absorbance observed in the presence of an isotype-matched mAb of irrelevant specificity (0% inhibition) and to the absorbance observed using excess test polyclonal antibodies (100% inhibition).

As another example, sera from a subject containing polyclonal antibodies may be obtained. The polyclonal antibodies (unlabeled) are then incubated with target antigen bound to a plate that has been blocked non-specifically with bovine serum albumin or a similar blocking reagent. Unlabeled non-specific antibodies (e.g., those present in sera from a naïve subject or an isotype-matched mAb of irrelevant specificity) are also incubated with the same target antigen bound to another plate that has also been blocked non-specifically with bovine serum albumin or a similar blocking reagent. The plates are then washed and the control antibody labeled with, for example, biotin is added to each. Following washing, horseradish peroxidase (HRP)-conjugated streptavidin is added to determine the amount of bound biotinylated control antibody. After addition of a substrate (TMB), the development of the enzymatic reaction is stopped with sulfuric acid. The absorbance of the two plates is then measured and compared. Any reduction in absorbance is indicative of the level of competitive inhibition.

“Couple” or “Coupled” or “Couples” (and the like) means to chemically associate one entity (for example a moiety) with another. In some embodiments, the coupling is covalent, meaning that the coupling occurs in the context of the presence of a covalent bond between the two entities. In non-covalent embodiments, the non-covalent coupling is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. In embodiments, encapsulation is a form of coupling.

“Concomitantly” means administering two or substances to a subject in a manner that is correlated in time, preferably sufficiently correlated in time so as to provide a modulation in an immune response. In embodiments, concomitant administration may occur through administration of two or more substances in the same dosage form. In other embodiments, concomitant administration may encompass administration of two or more substances in different dosage forms, but within a specified period of time, preferably within 1 month, more preferably within 1 week, still more preferably within 1 day, and even more preferably within 1 hour.

“Derived” means taken from a source and subjected to substantial modification. For instance, a peptide or nucleic acid with a sequence with only 50% identity to a natural peptide or nucleic acid, preferably a natural consensus peptide or nucleic acid, would be said to be derived from the natural peptide or nucleic acid. Substantial modification is modification that significantly affects the chemical or immunological properties of the material in question. Derived peptides and nucleic acids can also include those with a sequence with greater than 50% identity to a natural peptide or nucleic acid sequence if said derived peptides and nucleic acids have altered chemical or immunological properties as compared to the natural peptide or nucleic acid. These chemical or immunological properties comprise hydrophilicity, stability, affinity, and ability to couple with a carrier such as a synthetic nanocarrier.

“Dosage form” means a pharmacologically and/or immunologically active material in a medium, carrier, vehicle, or device suitable for administration to a subject.

“Human influenza A virus M2 protein” or “HIAV M2” or “M2” means an influenza matrix protein 2 encoded by segment 7 of the influenza A virus genome. Human influenza A virus M2 protein is usually produced by translation from a mRNA derived from this viral genome segment. In some embodiments, M2 usually comprises 97 amino acids.

“Ectodomain region of human influenza A virus M2 protein” or “Ectodomain of M2” or “M2 Ectodomain” or “HIAV M2 Ectodomain” or “M2e” means the N-terminal externally exposed domain (ectodomain) of HIAV M2 usually comprising 23 or 24 amino acids (in the 23-mer case the N-terminal methionine is absent) In an embodiment, a peptide obtained or derived from the ectodomain region of human influenza A virus M2 protein comprises a peptide obtained or derived from one or more of the following sequences (W. Kowalczyk, et al; Bioconjugate Chem. 2010, 21:102-110):

TABLE 1

| Representative | Sub- | | SEQ |
|---|---|---|---|
| virus | type | Amino acid sequences * | ID |
| A/Wilson-Smith/1933 | H1N1 | MSLLTEVETPIRNEWGCRCNDSSD | 1 |
| A/Puerto Rico/8/34 | H1N1 | MSLLTEVETPIRNEWGCRCNGSSD | 2 |
| A/Wisconsin/3523/88 | H1N1 | 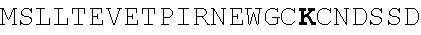 | 3 |
| A/California/04/2009 | H1N1 | 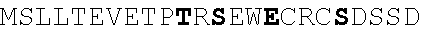 | 4 |
| A/Aichi/470/68 | H3N1 | MSLLTEVETPIRNEWGCRCNDSSD | 5 |
| A/Hebei/19/95 | H3N2 | 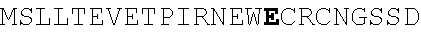 | 6 |
| A/Viet Nam/1203/2004 | H5N1 | 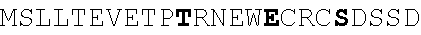 | 7 |
| A/Chicken/Nakorn- Patom/Thailand | H5N1 | 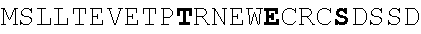 | 8 |
| A/Thailand/1KAN-1)/04 | H5N1 | 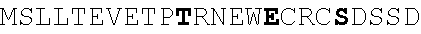 | 9 |
| A/Hong Kong/156/97 | H5N1 | 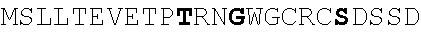 | 10 |
| A/Duck/1525/81 | H5N1 | 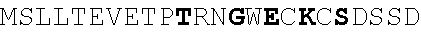 | 11 |
| A/Chicken/New York/95 | H7N2 | 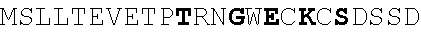 | 12 |
| Consensus | | MSLLTEVETPTRNEWESRSSDSSD | 13 |

In a preferred embodiment, the sequence used in creating peptides obtained or derived from M2e comprises:

(same as H5N1 from A/Viet Nam/1203/2004, SEQ ID NO: 14)
H-Met-Ser-Leu-Leu-Thr-Glu-Val-Glu-Thr-Pro-Thr-Arg-Asn-Glu-Trp-Glu-Cys-Arg-
Cys-Ser-Asp-Ser-Ser-Asp.

In a preferred embodiment, the recited peptide comprises:

(SEQ ID NO: 15)
H-Met-Ser-Leu-Leu-Thr-Glu-Val-Glu-Thr-Pro-Thr-Arg-Asn-Glu-Trp-Glu-Cys-Arg-
Cys-Ser-Asp-Gly-Gly-propargylamide .

The above sequence is based on M2e from H5N1 from A/Viet Nam/1203/2004 wherein the Ser-Ser-Asp sequence is replaced with Gly-Gly-propargylamide at the C-terminal. This modification facilitates coupling of the peptide using CuAAC click chemistry, as is disclosed elsewhere herein.

“Encapsulate” means to enclose at least a portion of a substance within a synthetic nanocarrier. In some embodiments, a substance is enclosed completely within a synthetic nanocarrier. In other embodiments, most or all of a substance that is encapsulated is not exposed to the local environment external to the synthetic nanocarrier. In other embodiments, no more than 50%, 40%, 30%, 20%, 10% or 5% is exposed to the local environment. Encapsulation is distinct from absorption, which places most or all of a substance on a surface of a synthetic nanocarrier, and leaves the substance exposed to the local environment external to the synthetic nanocarrier.

“Isolated nucleic acid” means a nucleic acid that is separated from its native environment and present in sufficient quantity to permit its identification or use. An isolated nucleic acid may be one that is (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. Any of the nucleic acids provided herein may be isolated. Any of the antigens provided herein may be provided as a nucleic acid that encodes it, and such nucleic acid may also be isolated.

“Isolated peptide” means the peptide is separated from its native environment and present in sufficient quantity to permit its identification or use. This means, for example, the peptide may be (i) selectively produced by expression cloning or (ii) purified as by chromatography or electrophoresis. Isolated peptides may be, but need not be, substantially pure. Because an isolated peptide may be admixed with a pharmaceutically acceptable carrier in a pharmaceutical preparation, the peptide may comprise only a small percentage by weight of the preparation. The peptide is nonetheless isolated in that it has been separated from the substances with which it may be associated in living systems, i.e., isolated from other proteins, etc. Any of the peptides provided herein may be isolated.

“Maximum dimension of a synthetic nanocarrier” means the largest dimension of a nanocarrier measured along any axis of the synthetic nanocarrier. “Minimum dimension of a synthetic nanocarrier” means the smallest dimension of a synthetic nanocarrier measured along any axis of the synthetic nanocarrier. For example, for a spheriodal synthetic nanocarrier, the maximum and minimum dimension of a synthetic nanocarrier would be substantially identical, and would be the size of its diameter. Similarly, for a cuboidal synthetic nanocarrier, the minimum dimension of a synthetic nanocarrier would be the smallest of its height, width or length, while the maximum dimension of a synthetic nanocarrier would be the largest of its height, width or length. In an embodiment, a minimum dimension of at least 75%, preferably at least 80%, more preferably at least 90%, of the synthetic nanocarriers in a sample, based on the total number of synthetic nanocarriers in the sample, is greater than 100 nm. In an embodiment, a maximum dimension of at least 75%, preferably at least 80%, more preferably at least 90%, of the synthetic nanocarriers in a sample, based on the total number of synthetic nanocarriers in the sample, is equal to or less than 5 μm. Preferably, a minimum dimension of at least 75%, preferably at least 80%, more preferably at least 90%, of the synthetic nanocarriers in a sample, based on the total number of synthetic nanocarriers in the sample, is greater than 110 nm, more preferably greater than 120 nm, more preferably greater than 130 nm, and more preferably still greater than 150 nm. Aspects ratios of the maximum and minimum dimensions of inventive synthetic nanocarriers may vary depending on the embodiment. For instance, aspect ratios of the maximum to minimum dimensions of the synthetic nanocarriers may vary from 1:1 to 1,000,000:1, preferably from 1:1 to 100,000:1, more preferably from 1:1 to 1000:1, still preferably from 1:1 to 100:1, and yet more preferably from 1:1 to 10:1. Preferably, a maximum dimension of at least 75%, preferably at least 80%, more preferably at least 90%, of the synthetic nanocarriers in a sample, based on the total number of synthetic nanocarriers in the sample is equal to or less than 3 μm, more preferably equal to or less than 2 μm, more preferably equal to or less than 1 μm, more preferably equal to or less than 800 nm, more preferably equal to or less than 600 nm, and more preferably still equal to or less than 500 nm. In preferred embodiments, a maximum dimension of at least 75%, preferably at least 80%, more preferably at least 90%, of the synthetic nanocarriers in a sample, based on the total number of synthetic nanocarriers in the sample, is equal to or greater than 100 nm, more preferably equal to or greater than 120, more preferably equal to or greater than 130 nm, more preferably equal to or greater than 140 nm, and more preferably still equal to or greater than 150 nm. Measurement of synthetic nanocarrier sizes is obtained by suspending the synthetic nanocarriers in a liquid (usually aqueous) media and using dynamic light scattering (DLS) (e.g. using a Brookhaven ZetaPALS instrument). For example, a suspension of synthetic nanocarriers can be diluted from an aqueous buffer into purified water to achieve a final synthetic nanocarrier suspension concentration of approximately 0.01 to 0.1 mg/mL. The diluted suspension may be prepared directly inside, or transferred to, a suitable cuvette for DLS analysis. The cuvette may then be placed in the DLS, allowed to equilibrate to the controlled temperature, and then scanned for sufficient time to aquire a stable and reproducible distribution based on appropriate inputs for viscosity of the medium and refractive indicies of the sample. The effective diameter, or mean of the distribution, is then reported.

“Obtained” means taken from a source without substantial modification. Substantial modification is modification that significantly affects the chemical or immunological properties of the material in question. For example, as a non-limiting example, a peptide or nucleic acid with a sequence with greater than 90%, preferably greater than 95%, preferably greater than 97%, preferably greater than 98%, preferably greater than 99%, preferably 100%, identity to a natural peptide or nucleotide sequence, preferably a natural consensus peptide or nucleotide sequence, and chemical and/or immunological properties that are not significantly different from the natural peptide or nucleic acid would be said to be obtained from the natural peptide or nucleotide sequence. These chemical or immunological properties comprise hydrophilicity, stability, affinity, and ability to couple with a carrier such as a synthetic nanocarrier.

“Peptide” means a compound comprising between 2 and 100 amino acids. Peptides according to the invention may be obtained or derived from a variety of sources, preferably from human influenza A virus M2 protein.

“Pharmaceutically acceptable excipient” means a pharmacologically inactive material used together with the recited synthetic nanocarriers to formulate the inventive compositions. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers.

“Subject” means animals, including warm blooded mammals such as humans and primates; avians; domestic household or farm animals such as cats, dogs, sheep, goats, cattle, horses and pigs; laboratory animals such as mice, rats and guinea pigs; fish; reptiles; zoo and wild animals; and the like.

“Synthetic nanocarrier(s)” means a discrete object that is not found in nature, and that possesses at least one dimension that is less than or equal to 5 microns in size. Albumin nanoparticles are generally included as synthetic nanocarriers, however in certain embodiments the synthetic nanocarriers do not comprise albumin nanoparticles. In some embodiments, inventive synthetic nanocarriers do not comprise chitosan. In some embodiments, synthetic nanocarriers are present in an amount sufficient to provide an immune response to the peptide upon administration of the composition to a subject. In some embodiments, amounts of the synthetic nanocarriers may range from 0.1 micrograms to 500 micrograms, preferably from 1 micrograms to 100 micrograms.

A synthetic nanocarrier can be, but is not limited to, one or a plurality of lipid-based nanoparticles, polymeric nanoparticles, metallic nanoparticles, surfactant-based emulsions, dendrimers, buckyballs, nanowires, virus-like particles, peptide or protein-based particles (such as albumin nanoparticles) and/or nanoparticles that are developed using a combination of nanomaterials such as lipid-polymer nanoparticles. Synthetic nanocarriers may be a variety of different shapes, including but not limited to spheroidal, cuboidal, pyramidal, oblong, cylindrical, toroidal, and the like. Synthetic nanocarriers according to the invention comprise one or more surfaces. Exemplary synthetic nanocarriers that can be adapted for use in the practice of the present invention comprise: (1) the biodegradable nanoparticles disclosed in U.S. Pat. No. 5,543,158 to Gref et al., (2) the polymeric nanoparticles of Published US Patent Application 20060002852 to Saltzman et al., (3) the lithographically constructed nanoparticles of Published US Patent Application 20090028910 to DeSimone et al., (4) the disclosure of WO 2009/051837 to von Andrian et al., or (5) the nanoparticles disclosed in Published US Patent Application 2008/0145441 to Penades et al., (6) the protein nanoparticles disclosed in Published US Patent Application 20090226525 to de los Rios et al., (7) the virus-like particles disclosed in published US Patent Application 20060222652 to Sebbel et al., (8) the nucleic acid coupled virus-like particles disclosed in published US Patent Application 20060251677 to Bachmann et al., (9) the virus-like particles disclosed in WO2010047839A1 or WO2009106999A2, or (10) the nanoprecipitated nanoparticles disclosed in P. Paolicelli et al., “Surface-modified PLGA-based Nanoparticles that can Efficiently Associate and Deliver Virus-like Particles” Nanomedicine. 5(6):843-853 (2010). In embodiments, synthetic nanocarriers may possess an aspect ratio greater than 1:1, 1:1.2, 1:1.5, 1:2, 1:3, 1:5, 1:7, or greater than 1:10.

Synthetic nanocarriers according to the invention that have a minimum dimension of equal to or less than about 100 nm, preferably equal to or less than 100 nm, do not comprise a surface with hydroxyl groups that activate complement or alternatively comprise a surface that consists essentially of moieties that are not hydroxyl groups that activate complement. In a preferred embodiment, synthetic nanocarriers according to the invention that have a minimum dimension of equal to or less than about 100 nm, preferably equal to or less than 100 nm, do not comprise a surface that substantially activates complement or alternatively comprise a surface that consists essentially of moieties that do not substantially activate complement. In a more preferred embodiment, synthetic nanocarriers according to the invention that have a minimum dimension of equal to or less than about 100 nm, preferably equal to or less than 100 nm, do not comprise a surface that activates complement or alternatively comprise a surface that consists essentially of moieties that do not activate complement. In embodiments, synthetic nanocarriers exclude virus-like particles. In embodiments, when synthetic nanocarriers comprise virus-like particles, the virus-like particles comprise non-natural adjuvant (meaning that the VLPs comprise an adjuvant other than naturally occurring RNA generated during the production of the VLPs). In embodiments, synthetic nanocarriers may possess an aspect ratio greater than 1:1, 1:1.2, 1:1.5, 1:2, 1:3, 1:5, 1:7, or greater than 1:10.

“T cell antigen” means any antigen that is recognized by and triggers an immune response in a T cell (e.g., an antigen that is specifically recognized by a T cell receptor on a T cell or an NKT cell via presentation of the antigen or portion thereof bound to a Class I or Class II major histocompatability complex molecule (MHC), or bound to a CD1 complex. In some embodiments, an antigen that is a T cell antigen is also a B cell antigen. In other embodiments, the T cell antigen is not also a B cell antigen. T cell antigens generally are proteins or peptides. T cell antigens may be an antigen that stimulates a CD8+ T cell response, a CD4+ T cell response, or both. The nanocarriers, therefore, in some embodiments can effectively stimulate both types of responses.

In some embodiments the T cell antigen is a T helper cell antigen (i.e. one that can generate an enhanced response to a B cell antigen, preferably an unrelated B cell antigen, through stimulation of T cell help). In embodiments, a T helper cell antigen may comprise one or more peptides obtained or derived from tetanus toxoid, Epstein-Barr virus, influenza virus, respiratory syncytial virus, measles virus, mumps virus, rubella virus, cytomegalovirus, adenovirus, diphtheria toxoid, or a PADRE peptide (known from the work of Sette et al. U.S. Pat. No. 7,202,351). In other embodiments, a T helper cell antigen may comprise one or more lipids, or glycolipids, including but not limited to: α-galactosylceramide (α-GalCer), α-linked glycosphingolipids (from Sphingomonas spp.), galactosyl diacylglycerols (from Borrelia burgdorferi), lypophosphoglycan (from Leishmania donovani), and phosphatidylinositol tetramannoside (PIM4) (from Mycobacterium leprae). For additional lipids and/or glycolipids useful as a T helper cell antigen, see V. Cerundolo et al., “Harnessing invariant NKT cells in vaccination strategies.” Nature Rev Immun, 9:28-38 (2009). In embodiments, CD4+ T-cell antigens may be derivatives of a CD4+ T-cell antigen that is obtained from a source, such as a natural source. In such embodiments, CD4+ T-cell antigen sequences, such as those peptides that bind to MHC II, may have at least 70%, 80%, 90%, or 95% identity to the antigen obtained from the source. In embodiments, the T cell antigen, preferably a T helper cell antigen, may be coupled to, or uncoupled from, a synthetic nanocarrier.

“Vaccine” means a composition of matter that improves the immune response to a particular pathogen or disease. A vaccine typically contains factors that stimulate a subject's immune system to recognize a specific antigen as foreign and eliminate it from the subject's body. A vaccine also establishes an immunologic ‘memory’ so the antigen will be quickly recognized and responded to if a person is re-challenged. Vaccines can be prophylactic (for example to prevent future infection by any pathogen), or therapeutic (for example a vaccine against a tumor specific antigen for the treatment of cancer). In embodiments, a vaccine may comprise dosage forms according to the invention.

### III. Inventive Compositions

In some embodiments, a peptide obtained or derived from the ectodomain region of human influenza A virus M2 protein comprises a peptide obtained or derived from one or more of the sequences listed in Table 1 or FIG. 5. In some embodiments wherein the recited peptide is obtained or derived from M2e, modifications that can be made to the M2e sequences comprise c-terminus or n-terminus addition of a linker group to enhance coupling between the peptide and synthetic nanocarriers (e.g. addition of a terminal alkyne or azide for use in CuAAC “click” reactions); reduction in peptide length; replacement of internal Cys residues by Ser residues to avoid synthetic problems during conjugation (for example, when C-terminal Cys group is used for coupling to synthetic nanocarriers).

A wide variety of synthetic nanocarriers can be used according to the invention. In some embodiments, synthetic nanocarriers are spheres or spheroids. In some embodiments, synthetic nanocarriers are flat or plate-shaped. In some embodiments, synthetic nanocarriers are cubes or cubic. In some embodiments, synthetic nanocarriers are ovals or ellipses. In some embodiments, synthetic nanocarriers are cylinders, cones, or pyramids.

In some embodiments, it is desirable to use a population of synthetic nanocarriers that is relatively uniform in terms of size, shape, and/or composition so that each synthetic nanocarrier has similar properties. For example, at least 80%, at least 90%, or at least 95% of the synthetic nanocarriers, based on the total number of synthetic nanocarriers, may have a minimum dimension or maximum dimension that falls within 5%, 10%, or 20% of the average diameter or average dimension of the synthetic nanocarriers. In some embodiments, a population of synthetic nanocarriers may be heterogeneous with respect to size, shape, and/or composition.

Synthetic nanocarriers can be solid or hollow and can comprise one or more layers. In some embodiments, each layer has a unique composition and unique properties relative to the other layer(s). To give but one example, synthetic nanocarriers may have a core/shell structure, wherein the core is one layer (e.g. a polymeric core) and the shell is a second layer (e.g. a lipid bilayer or monolayer). Synthetic nanocarriers may comprise a plurality of different layers.

In some embodiments, synthetic nanocarriers may optionally comprise one or more lipids. In some embodiments, a synthetic nanocarrier may comprise a liposome. In some embodiments, a synthetic nanocarrier may comprise a lipid bilayer. In some embodiments, a synthetic nanocarrier may comprise a lipid monolayer. In some embodiments, a synthetic nanocarrier may comprise a micelle. In some embodiments, a synthetic nanocarrier may comprise a core comprising a polymeric matrix surrounded by a lipid layer (e.g., lipid bilayer, lipid monolayer, etc.). In some embodiments, a synthetic nanocarrier may comprise a non-polymeric core (e.g., metal particle, quantum dot, ceramic particle, bone particle, viral particle, proteins, nucleic acids, carbohydrates, etc.) surrounded by a lipid layer (e.g., lipid bilayer, lipid monolayer, etc.).

In some embodiments, synthetic nanocarriers can comprise one or more polymers. In some embodiments, such a polymer can be surrounded by a coating layer (e.g., liposome, lipid monolayer, micelle, etc.). In some embodiments, various elements of the synthetic nanocarriers can be coupled with the polymer.

In some embodiments, an immunofeature surface, targeting moiety, and/or oligonucleotide (or other element) can be covalently associated with a polymeric matrix. In some embodiments, covalent association is mediated by a linker. In some embodiments, an immunofeature surface, targeting moiety, and/or oligonucleotide (or other element) can be noncovalently associated with a polymeric matrix. For example, in some embodiments, an immunofeature surface, targeting moiety, and/or oligonucleotide (or other element) can be encapsulated within, surrounded by, and/or dispersed throughout a polymeric matrix. Alternatively or additionally, an immunofeature surface, targeting moiety, and/or nucleotide (or other element) can be associated with a polymeric matrix by hydrophobic interactions, charge interactions, van der Waals forces, etc.

A wide variety of polymers and methods for forming such polymers are known conventionally. In general, a polymeric synthetic nanocarrier comprises one or more polymers. Polymers may be natural or unnatural (synthetic) polymers. Polymers may be homopolymers or copolymers comprising two or more monomers. In terms of sequence, copolymers may be random, block, or comprise a combination of random and block sequences. Typically, polymers in accordance with the present invention are organic polymers.

Examples of polymers suitable for use in the present invention include, but are not limited to polyethylenes, polycarbonates (e.g. poly(1,3-dioxan-2one)), polyanhydrides (e.g. poly(sebacic anhydride)), polypropylfumerates, polyamides (e.g. polycaprolactam), polyacetals, polyethers, polyesters (e.g., polylactide, polyglycolide, polylactide-co-glycolide, polycaprolactone, polyhydroxyacid (e.g. poly(β-hydroxyalkanoate))), poly(orthoesters), polycyanoacrylates, polyvinyl alcohols, polyurethanes, polyphosphazenes, polyacrylates, polymethacrylates, polyureas, polystyrenes, and polyamines, polylysine, polylysine-PEG copolymers, and poly(ethyleneimine), poly(ethylene imine)-PEG copolymers.

In some embodiments, polymers in accordance with the present invention include polymers which have been approved for use in humans by the U.S. Food and Drug Administration (FDA) under 21 C.F.R. §177.2600, including but not limited to polyesters (e.g., polylactic acid, poly(lactic-co-glycolic acid), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments, polymers can be hydrophilic. For example, polymers may comprise anionic groups (e.g., phosphate group, sulphate group, carboxylate group); cationic groups (e.g., quaternary amine group); or polar groups (e.g., hydroxyl group, thiol group, amine group). In some embodiments, a synthetic nanocarrier comprising a hydrophilic polymeric generates a hydrophilic environment within the synthetic nanocarrier. In some embodiments, polymers can be hydrophobic. In some embodiments, a synthetic nanocarrier comprising a hydrophobic polymeric generates a hydrophobic environment within the synthetic nanocarrier. Selection of the hydrophilicity or hydrophobicity of the polymer may have an impact on the nature of materials that are incorporated (e.g. coupled) within the synthetic nanocarrier.

In some embodiments, polymers may be modified with one or more moieties and/or functional groups. A variety of moieties or functional groups can be used in accordance with the present invention. In some embodiments, polymers may be modified with polyethylene glycol (PEG), with a carbohydrate, and/or with acyclic polyacetals derived from polysaccharides (Papisov, 2001, ACS Symposium Series, 786:301). Certain embodiments may be made using the general teachings of U.S. Pat. No. 5,543,158 to Gref et al., or WO publication WO2009/051837 by Von Andrian et al.

In some embodiments, polymers may be modified with a lipid or fatty acid group. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEG copolymers and copolymers of lactide and glycolide (e.g., PLA-PEG copolymers, PGA-PEG copolymers, PLGA-PEG copolymers, and derivatives thereof. In some embodiments, polyesters include, for example, poly(caprolactone), poly(caprolactone)-PEG copolymers, poly(L-lactide-co-L-lysine), poly(serine ester), poly(4-hydroxy-L-proline ester), poly[α-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids (e.g. DNA, or derivatives thereof). Amine-containing polymers such as poly(lysine) (Zauner et al., 1998, Adv. Drug Del. Rev., 30:97; and Kabanov et al., 1995, Bioconjugate Chem., 6:7), poly(ethylene imine) (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372) are positively-charged at physiological pH, form ion pairs with nucleic acids, and mediate transfection in a variety of cell lines. In some embodiments, the inventive synthetic nanocarriers may not comprise (or may exclude) cationic polymers.

In some embodiments, polymers can be degradable polyesters bearing cationic side chains (Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-L-lysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633), and poly(4-hydroxy-L-proline ester) (Putnam et al., 1999, Macromolecules, 32:3658; and Lim et al., 1999, J. Am. Chem. Soc., 121:5633).

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing certain suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

In some embodiments, polymers can be linear or branched polymers. In some embodiments, polymers can be dendrimers. In some embodiments, polymers can be substantially cross-linked to one another. In some embodiments, polymers can be substantially free of cross-links. In some embodiments, polymers can be used in accordance with the present invention without undergoing a cross-linking step. It is further to be understood that inventive synthetic nanocarriers may comprise block copolymers, graft copolymers, blends, mixtures, and/or adducts of any of the foregoing and other polymers. Those skilled in the art will recognize that the polymers listed herein represent an exemplary, not comprehensive, list of polymers that can be of use in accordance with the present invention.

In some embodiments, synthetic nanocarriers do not comprise a polymeric component. In some embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

In some embodiments, synthetic nanocarriers may optionally comprise one or more amphiphilic entities. In some embodiments, an amphiphilic entity can promote the production of synthetic nanocarriers with increased stability, improved uniformity, or increased viscosity. In some embodiments, amphiphilic entities can be associated with the interior surface of a lipid membrane (e.g., lipid bilayer, lipid monolayer, etc.). Many amphiphilic entities known in the art are suitable for use in making synthetic nanocarriers in accordance with the present invention. Such amphiphilic entities include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid monoglycerides; fatty acid diglycerides; fatty acid amides; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl stearate; isopropyl myristate; tyloxapol; poly(ethylene glycol)5000-phosphatidylethanolamine; poly(ethylene glycol)400-monostearate; phospholipids; synthetic and/or natural detergents having high surfactant properties; deoxycholates; cyclodextrins; chaotropic salts; ion pairing agents; and combinations thereof. An amphiphilic entity component may be a mixture of different amphiphilic entities. Those skilled in the art will recognize that this is an exemplary, not comprehensive, list of substances with surfactant activity. Any amphiphilic entity may be used in the production of synthetic nanocarriers to be used in accordance with the present invention.

In some embodiments, synthetic nanocarriers may optionally comprise one or more carbohydrates. Carbohydrates may be natural or synthetic. A carbohydrate may be a derivatized natural carbohydrate. In certain embodiments, a carbohydrate comprises monosaccharide or disaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is a polysaccharide, including but not limited to pullulan, cellulose, microcrystalline cellulose, hydroxypropyl methylcellulose (HPMC), hydroxycellulose (HC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan. In some embodiments, the inventive synthetic nanocarriers do not comprise (or specifically exclude) carbohydrates, such as a polysaccharide. In certain embodiments, the carbohydrate may comprise a carbohydrate derivative such as a sugar alcohol, including but not limited to mannitol, sorbitol, xylitol, erythritol, maltitol, and lactitol.

In some embodiments, dosage forms according to the invention may comprise inventive synthetic nanocarriers in combination with pharmaceutically acceptable excipients, such as preservatives, buffers, saline, or phosphate buffered saline. The dosage forms may be made using conventional pharmaceutical manufacturing and compounding techniques. Inventive dosage forms may comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). In an embodiment, inventive synthetic nanocarriers are suspended in sterile saline solution for injection together with a preservative.

In embodiments, when preparing synthetic nanocarriers as carriers for adjuvants for use in vaccines, methods for coupling the adjuvants to the synthetic nanocarriers may be useful. If the adjuvant is a small molecule it may be of advantage to attach the adjuvant to a polymer prior to the assembly of the synthetic nanocarriers. In embodiments, it may also be an advantage to prepare the synthetic nanocarriers with surface groups that are used to couple the adjuvant to the synthetic nanocarrier through the use of these surface groups rather than attaching the adjuvant to a polymer and then using this polymer conjugate in the construction of synthetic nanocarriers.

The recited peptides can be coupled to the synthetic nanocarriers by a variety of methods. In some embodiments, the peptide is coupled to an external surface of the synthetic nanocarrier covalently or non-covalently, preferably though its C terminus or its N-terminus, more preferably the peptide is coupled through its C terminus to the external surface.

In certain embodiments, the coupling can be a covalent linker. In some embodiments, peptides according to the invention can be covalently coupled to the external surface via a 1,2,3-triazole linker formed by the 1,3-dipolar cycloaddition reaction of azido groups on the surface of the nanocarrier with peptides containing an alkyne group or by the 1,3-dipolar cycloaddition reaction of alkynes on the surface of the nanocarrier with peptides containing an azido group. Such cycloaddition reactions are preferably performed in the presence of a Cu(I) catalyst along with a suitable Cu(I)-ligand and a reducing agent to reduce Cu(II) compound to catalytic active Cu(I) compound. This Cu(I)-catalyzed azide-alkyne cycloaddition (CuAAC) can also be referred as the click reaction.

Additionally, the covalent coupling may comprise a covalent linker that comprises an amide linker, a disulfide linker, a thioether linker, a hydrazone linker, a hydrazide linker, an imine or oxime linker, an urea or thiourea linker, an amidine linker, an amine linker, and a sulfonamide linker.

An amide linker is formed via an amide bond between an amine on one component such as the peptide with the carboxylic acid group of a second component such as the nanocarrier. The amide bond in the linker can be made using any of the conventional amide bond forming reactions with suitably protected amino acids or peptides and activated carboxylic acid such N-hydroxysuccinimide-activated ester.

A disulfide linker is made via the formation of a disulfide (S—S) bond between two sulfur atoms of the form, for instance, of R₁—S—S—R₂. A disulfide bond can be formed by thiol exchange of a peptide containing thiol/mercaptan group (—SH) with another activated thiol group on a polymer or nanocarrier or a nanocarrier containing thiol/mercaptan groups with a peptide containing activated thiol group.

A triazole linker, specifically a 1,2,3-triazole of the form

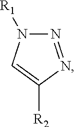

wherein R₁ and R₂ may be any chemical entities, is made by the 1,3-dipolar cycloaddition reaction of an azide attached to a first component such as the nanocarrier with a terminal alkyne attached to a second component such as the peptide. The 1,3-dipolar cycloaddition reaction is performed with or without a catalyst, preferably with Cu(I)-catalyst, which links the two components through a 1,2,3-triazole function. This chemistry is described in detail by Sharpless et al., Angew. Chem. Int. Ed. 41(14), 2596, (2002) and Meldal, et al, Chem. Rev., 2008, 108(8), 2952-3015 and is often referred to as a “click” reaction or CuAAC.

In some embodiments, a polymer containing an azide or alkyne group, terminal to the polymer chain is prepared. This polymer is then used to prepare a synthetic nanocarrier in such a manner that a plurality of the alkyne or azide groups are positioned on the surface of that nanocarrier. Alternatively, the synthetic nanocarrier can be prepared by another route, and subsequently functionalized with alkyne or azide groups. The peptide is prepared with the presence of either an alkyne (if the polymer contains an azide) or an azide (if the polymer contains an alkyne) group. The peptide is then allowed to react with the nanocarrier via the 1,3-dipolar cycloaddition reaction with or without a catalyst which covalently couples the peptide to the particle through the 1,4-disubstituted 1,2,3-triazole linker.

A thioether linker is made by the formation of a sulfur-carbon (thioether) bond in the form, for instance, of R₁—S—R₂. Thioether can be made by either alkylation of a thiol/mercaptan (—SH) group on one component such as the peptide with an alkylating group such as halide or epoxide on a second component such as the nanocarrier. Thioether linkers can also be formed by Michael addition of a thiol/mercaptan group on one component such as a peptide to an electron-deficient alkene group on a second component such as a polymer containing a maleimide group or vinyl sulfone group as the Michael acceptor. In another way, thioether linkers can be prepared by the radical thiol-ene reaction of a thiol/mercaptan group on one component such as a peptide with an alkene group on a second component such as a polymer or nanocarrier.

A hydrazone linker is made by the reaction of a hydrazide group on one component such as the peptide with an aldehyde/ketone group on the second component such as the nanocarrier.

A hydrazide linker is formed by the reaction of a hydrazine group on one component such as the peptide with a carboxylic acid group on the second component such as the nanocarrier. Such reaction is generally performed using chemistry similar to the formation of amide bond where the carboxylic acid is activated with an activating reagent.

An imine or oxime linker is formed by the reaction of an amine or N-alkoxyamine (or aminooxy) group on one component such as the peptide with an aldehyde or ketone group on the second component such as the nanocarrier.

An urea or thiourea linker is prepared by the reaction of an amine group on one component such as the peptide with an isocyanate or thioisocyanate group on the second component such as the nanocarrier.

An amidine linker is prepared by the reaction of an amine group on one component such as the peptide with an imidoester group on the second component such as the nanocarrier.

An amine linker is made by the alkylation reaction of an amine group on one component such as the peptide with an alkylating group such as halide, epoxide, or sulfonate ester group on the second component such as the nanocarrier. Alternatively, an amine linker can also be made by reductive amination of an amine group on one component such as the peptide with an aldehyde or ketone group on the second component such as the nanocarrier with a suitable reducing reagent such as sodium cyanoborohydride or sodium triacetoxyborohydride.

A sulfonamide linker is made by the reaction of an amine group on one component such as the peptide with a sulfonyl halide (such as sulfonyl chloride) group on the second component such as the nanocarrier.

A sulfone linker is made by Michael addition of a nucleophile to a vinyl sulfone. Either the vinyl sulfone or the nucleophile may be on the surface of the nanoparticle or attached to the antigen.

Additional descriptions of available conjugation methods are available in “Bioconjugate Techniques”, 2nd Edition By Greg T. Hermanson, Published by Academic Press, Inc., 2008) (Hermanson 2008.)

The peptide can also be conjugated to the nanocarrier via non-covalent conjugation methods. For examples, a negative charged peptide can be conjugated to a positive charged nanocarrier through electrostatic adsorption. A peptide containing a metal ligand can also be conjugated to a nanocarrier containing a metal complex via a metal-ligand complex.

In some embodiments, an antigen can be attached to a polymer, for example polylactic acid-block-polyethylene glycol, prior to the assembly of the synthetic nanocarrier or the synthetic nanocarrier can be formed with reactive or activatable groups on its surface. In the latter case, the peptide is prepared with a group that is compatible with the attachment chemistry that is presented by the synthetic nanocarriers' surface. In other embodiments, a peptide antigen can be attached to VLPs or liposomes using a suitable linker. A linker is a compound or reagent that capable of coupling two molecules together. In an embodiment, the linker can be a homobifuntional or heterobifunctional reagent as described in Hermanson 2008. For example, a VLP or liposome synthetic nanocarrier containing a carboxylic group on the surface can be treated with a homobifunctional linker, adipic dihydrazide (ADH), in the presence of EDC to form the corresponding synthetic nanocarrier with the ADH linker. The resulting ADH linked synthetic nanocarrier is then conjugated with a peptide antigen containing an acid group via the other end of the ADH linker on NC to produce the corresponding VLP or liposome peptide conjugate.

In the present embodiments, a peptide obtained or derived from M2e according to the invention that comprises a C-terminal alkyne group may be conjugated via the Cu(I)-catalyzed 1,3-dipolar cycloaddition (CuAAC) to synthetic nanocarriers made of PLA-PEG-azide polymer while the azide groups are on the surface of the synthetic nanocarriers. In a specific embodiment, the Cu(I) catalyst is formed in situ from CuSO4 and sodium ascorbate. Preferably, a suitable Cu(I) ligand such as Tris(3-hydroxypropyltriazolylmethyl)amine, is used to maintain the activity of the Cu(I) catalyst. The reaction is performed in buffered aq solution (pH 6-9) at 4 to 25 C over 2-48 h.

In addition to covalent attachment the peptide can be adsorbed to a pre-formed synthetic nanocarrier or it can be encapsulated during the formation of the synthetic nanocarrier.

In some embodiments, the inventive synthetic nanocarriers may be coupled to one or more adjuvants, and/or may be coupled to a T-helper antigen. Types of adjuvants and T-helper antigens useful in the practice of the present invention have been described elsewhere. The amounts of such adjuvants and/or T-helper antigens to be included in the inventive synthetic nanocarriers may be determined using conventional dose ranging techniques. Adjuvants and/or T-helper antigens may be coupled to the synthetic nanocarriers using coupling methods disclosed elsewhere herein, or known conventionally, and adapted for use with the particular adjuvant and/or T-helper antigen (e.g. use of linker chemistries noted for use with the recited peptides obtained or derived from M2e, including the techniques of Hermanson 2008, or non-covalent coupling techniques (encapsulation, adsorption, and the like), etc., in each case adapted to the adjuvant and/or T-helper antigen of interest may also be used). Use of adjuvants and/or T-helper antigens can provide an improved immune response to the recited peptides obtained or derived from M2e.

### Methods of Making and Using the Inventive Compositions and Related Methods

Synthetic nanocarriers may be prepared using a wide variety of methods known in the art. For example, synthetic nanocarriers can be formed by methods as nanoprecipitation, flow focusing fluidic channels, spray drying, single and double emulsion solvent evaporation, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanomaterials have been described (Pellegrino et al., 2005, Small, 1:48; Murray et al., 2000, Ann. Rev. Mat. Sci., 30:545; and Trindade et al., 2001, Chem. Mat., 13:3843). Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, δ: 275; and Mathiowitz et al., 1988, J. Appl. Polymer Sci., 35:755, and also U.S. Pat. Nos. 5,578,325 and 6,007,845); P. Paolicelli et al., “Surface-modified PLGA-based Nanoparticles that can Efficiently Associate and Deliver Virus-like Particles” Nanomedicine. 5(6):843-853 (2010)).

Various materials may be coupled through encapsulation into synthetic nanocarriers as desirable using a variety of methods including but not limited to C. Astete et al., “Synthesis and characterization of PLGA nanoparticles” J. Biomater. Sci. Polymer Edn, Vol. 17, No. 3, pp. 247-289 (2006); K. Avgoustakis “Pegylated Poly(Lactide) and Poly(Lactide-Co-Glycolide) Nanoparticles: Preparation, Properties and Possible Applications in Drug Delivery” Current Drug Delivery 1:321-333 (2004); C. Reis et al., “Nanoencapsulation I. Methods for preparation of drug-loaded polymeric nanoparticles” Nanomedicine 2:8-21 (2006); P. Paolicelli et al., “Surface-modified PLGA-based Nanoparticles that can Efficiently Associate and Deliver Virus-like Particles” Nanomedicine. 5(6):843-853 (2010)). Other methods suitable for encapsulating materials, such as oligonucleotides, into synthetic nanocarriers may be used, including without limitation methods disclosed in U.S. Pat. No. 6,632,671 to Unger (Oct. 14, 2003).

In certain embodiments, synthetic nanocarriers are prepared by a nanoprecipitation process or spray drying. Conditions used in preparing synthetic nanocarriers may be altered to yield particles of a desired size or property (e.g., hydrophobicity, hydrophilicity, external morphology, “stickiness,” shape, etc.). The method of preparing the synthetic nanocarriers and the conditions (e.g., solvent, temperature, concentration, air flow rate, etc.) used may depend on the materials to be coupled to the synthetic nanocarriers and/or the composition of the polymer matrix.

If particles prepared by any of the above methods have a size range outside of the desired range, particles can be sized, for example, using a sieve.

Elements of the inventive synthetic nanocarriers—such as moieties of which an immunofeature surface is comprised, targeting moieties, polymeric matrices, antigens and the like—may be coupled to the overall synthetic nanocarrier, e.g., by one or more covalent bonds, or may be coupled by means of one or more linkers. Additional methods of functionalizing synthetic nanocarriers may be adapted from Published US Patent Application 2006/0002852 to Saltzman et al., Published US Patent Application 2009/0028910 to DeSimone et al., or Published International Patent Application WO/2008/127532 A1 to Murthy et al.

Alternatively or additionally, synthetic nanocarriers can be coupled to immunofeature surfaces, targeting moieties, adjuvants, various antigens, and/or other elements directly or indirectly via non-covalent interactions. In non-covalent embodiments, the non-covalent coupling is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. Such couplings may be arranged to be on an external surface or an internal surface of an inventive synthetic nanocarrier. In some embodiments, encapsulation and/or absorbtion are forms of coupling.

Doses of dosage forms contain varying amounts of synthetic nanocarriers and varying amounts of antigens, according to the invention. The amount of synthetic nanocarriers and/or antigens present in the inventive dosage forms can be varied according to the nature of the antigens, the therapeutic benefit to be accomplished, and other such parameters. In some embodiments, dose ranging studies can be conducted to establish optimal therapeutic amount of the synthetic nanocarriers and the amount of peptide antigens to be present in the dosage form. In some embodiments, the synthetic nanocarriers and the peptide antigens are present in the dosage form in an amount effective to generate an immune response to the peptide antigens upon administration to a subject. It is possible to determine amounts of the peptides effective to generate an immune response using conventional dose ranging studies and techniques in subjects. Inventive dosage forms may be administered at a variety of frequencies. In an embodiment, at least one administration of the dosage form is sufficient to generate a pharmacologically relevant response. In additional embodiments, at least two administrations, at least three administrations, or at least four administrations, of the dosage form are utilized to ensure a pharmacologically relevant response.

In embodiments, the inventive synthetic nanocarriers can be combined with other adjuvants by admixing in the same vehicle or delivery system. Such adjuvants may include, but are not limited to mineral salts, such as alum, alum combined with monphosphoryl lipid (MPL) A of Enterobacteria, such as Escherihia coli, Salmonella minnesota, Salmonella typhimurium, or Shigella flexneri or specifically with MPL® (AS04), MPL A of above-mentioned bacteria separately, saponins, such as QS-21, Quil-A, ISCOMs, ISCOMATRIX™, emulsions such as MF59™, Montanide® ISA 51 and ISA 720, AS02 (QS21+squalene+MPL®), liposomes and liposomal formulations such as AS01, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic® block co-polymers, specifically modified or prepared peptides, such as muramyl dipeptide, aminoalkyl glucosaminide 4-phosphates, such as RC529, or proteins, such as bacterial toxoids or toxin fragments. The doses of such other adjuvants can be determined using conventional dose ranging studies.

In embodiments, the inventive synthetic nanocarriers can be combined with an antigen different, similar or identical to those coupled to a nanocarrier (with or without adjuvant, utilizing or not utilizing another delivery vehicle) administered separately at a different time-point and/or at a different body location and/or by a different immunization route or with another antigen and/or adjuvant-carrying synthetic nanocarrier administered separately at a different time-point and/or at a different body location and/or by a different immunization route. In some embodiments, such antigen is another influenza antigen, for example, a neuraminidase, a surface antigen, a nucleocapsid protein, a matrix protein, a phosphoprotein, a fusion protein, a hemagglutinin, a hemagglutinin-neuraminidase, a glycoprotein capsular polysaccharides, a protein D, a M2 protein, or an antigenic fragment thereof, of an influenza virus.

In some embodiments, the inventive dosage forms may comprise the recited synthetic nanocarriers and one or more conventional influenza vaccines to form a multivalent influenza vaccine. This may be accomplished by simply admixing a dispersion comprising the recited synthetic nanocarriers with a solution or dispersion that comprises a conventional influenza vaccine. In an embodiment, the inventive dosage forms comprise the recited synthetic nanocarriers and influenza antigen that is not coupled to the recited synthetic nanocarriers.

Populations of synthetic nanocarriers may be combined to form pharmaceutical dosage forms according to the present invention using traditional pharmaceutical mixing methods. These include liquid-liquid mixing in which two or more suspensions, each containing one or more subset of nanocarriers, are directly combined or are brought together via one or more vessels containing diluent. As synthetic nanocarriers may also be produced or stored in a powder form, dry powder-powder mixing could be performed as could the re-suspension of two or more powders in a common media. Depending on the properties of the nanocarriers and their interaction potentials, there may be advantages conferred to one or another route of mixing.

In some embodiments, inventive synthetic nanocarriers are manufactured under sterile conditions or are terminally sterilized. This can ensure that resulting composition are sterile and non-infectious, thus improving safety when compared to non-sterile compositions. This provides a valuable safety measure, especially when subjects receiving synthetic nanocarriers have immune defects, are suffering from infection, and/or are susceptible to infection. In some embodiments, inventive synthetic nanocarriers may be lyophilized and stored in suspension or as lyophilized powder depending on the formulation strategy for extended periods without losing activity.

It is to be understood that the compositions of the invention can be made in any suitable manner, and the invention is in no way limited to compositions that can be produced using the methods described herein. Selection of an appropriate method may require attention to the properties of the particular moieties being associated.

The inventive compositions may be administered by a variety of routes of administration, including but not limited to intravenous, subcutaneous, pulmonary, intramuscular, intradermal, oral, intranasal, intramucosal, transmucosal, sublingual, rectal; ophthalmic, transdermal, transcutaneous or by a combination of these routes.

The compositions and methods described herein can be used to induce, enhance, suppress, modulate, direct, or redirect an immune response. The compositions and methods described herein can be used in the diagnosis, prophylaxis and/or treatment of conditions such as human influenza infections or other related disorders and/or conditions.

### EXAMPLES

### Example 1

### Synthetic Nanocarriers with Covalently Coupled M2e Peptide (Prophetic)

Synthetic nanocarriers are coupled to peptides obtained or derived from M2e using methods generally disclosed in Bioconjugate Chem. 2010, 21:102 as follows:

A peptide obtained or derived from M2e is prepared by solid-phase peptide synthesis:

Sequence:
(SEQ ID NO: 16)
H-Met-Ser-Leu-Leu-Thr-Glu-Val-Glu-Thr-Pro-Thr-Arg-Asn-Glu-Trp-
Glu-Ser-Arg-Ser-Ser-Asp-Ser-Ser-Asp-Cys.

The peptide sequence is based on the M2e of the virus A/Aichi/470/68 (H3N1): Met-Ser-Leu-Leu-Thr-Glu-Val-Glu-Thr-Pro-Ile-Arg-Asn-Glu-Trp-Gly-Cys-Arg-Cys-Asn-Asp-Ser-Ser-Asp-Aha-Cys-amide (SEQ ID NO: 17) where Aha (6-aminohexanoic acid) as a spacer was incorporated between the main M2 sequence and the C-terminal cysteine to minimize steric hindrance during the conjugation of the C-terminal cysteine thiol group with maleimide group on NCs.

Synthetic nanocarriers (NCs) are made by Water-oil-Water (WOW) double-emulsion evaporation process consisting of 25% wt of PLA-PEG-maleimide, made by ring opening polymerization of HO-PEG-maleimide with dl-lactide in the presence of Sn(Oct)₂, 50% wt of PLGA-R848 (a conjugate of poly-lactide-co-glycolide and resiquimod), 25% wt of polylactic acid (100□L2A) and ova peptide (as T-cell antigen, ovalbumin residues 323-339, sequence: H-Ile-Ser-Gln-Ala-Val-His-Ala-Ala-His-Ala-Glu-Ile-Asn-Glu-Ala-Gly-Arg-NH2 (SEQ ID NO: 18), acetate salt, Lot# B06395, prepared by Bachem Biosciences, Inc.). The loading of R848 and ova peptide is expected to be 2.9% wt of NCs and 0.9% wt of NCs, respectively.

Peptide is coupled to the NCs by Michael addition of the C-terminal thiol group in the M2e peptide to the surface maleimide group on the NCs as follows: The peptide (8 mg) is dissolved in 0.8 mL of PBS (pH 9) and mixed with 24 mg of NCs in 3 mL PBS (pH 9) at rt under argon in dark for 20-24 h. The NC-peptide suspension is then pellet-washed with PBS (pH 7.4, 2×8 mL) and re-suspended in 3 mL PBS (pH 7.4) for further analysis and bioassays.

### Example 2

### Synthetic Nanocarriers with Non-Covalently Coupled M2e Peptide (Prophetic)

Peptides obtained or derived from M2e peptide can be conjugated to gold synthetic nanocarriers by formation of the Au-thiol complex to give peptide-AuNC conjugates:

Step-1. Formation of AuNCs: An aq. solution of 500 mL of 1 mM HAuCl₄ is heated to reflux for 10 min with vigorous stirring in a 1 L round-bottom flask equipped with a condenser. A solution of 50 mL of 40 mM of trisodium citrate is then rapidly added to the stirring solution. The resulting deep wine red solution is kept at reflux for 25-30 min. The heat is then withdrawn and the solution is cooled to room temperature. The solution is then filtered through a 0.8 μm membrane filter to give the AuNCs in suspension. The AuNCs are characterized using visible spectroscopy and transmission electron microscopy. The AuNCs are ca. 20 nm diameter capped by citrate with peak absorption at 520 nm.

Step-2. Direct peptide coupling to AuNCs: A modified M2e peptide containing a C-terminal Cys group with the following sequence: H-Met-Ser-Leu-Leu-Thr-Glu-Val-Glu-Thr-Pro-Thr-Arg-Asn-Glu-Trp-Glu-Cys-Arg-Cys-Ser-Asp-Ser-Ser-Asp-Cys (SEQ ID NO: 19) is conjugated to the AuNCs made in Step 1 as follows: A solution of 145 μl of the peptide (10 μM in 10 mM pH 9.0 carbonate buffer) is added to 1 mL of 20 nm diameter citrate-capped gold nanoparticles (1.16 nM) to produce a molar ratio of c-terminal thiol to gold of 2500:1. The mixture is stirred at room temperature under argon for 1 hour to allow complete exchange of thiol with citrate on the gold nanoparticles. The peptide-AuNC conjugates are then purified by centrifugation at 12,000 g for 30 minutes. The supernatant is decanted and the pelleted peptide-AuNCs are resuspended in 1 mL WFI water for further analysis and bioassay.

### Example 3

### Synthetic Nanocarriers with Covalently Coupled M2e Peptide (Prophetic)

Virus-like particles (VLPSs) from Cowpea mosaic virus or tobacco mosaic virus (in 20 mM HEPES, 150 mM NaCl, pH 7.2) are derivatized by incubation with a 10-fold molar excess of cross-linker, succinimidyl-6-(beta-maleimidopropionamido)hexanoate at room temperature for 2-4 h. After removal of free cross-linker by extensive dialysis against 20 mM HEPES, 150 mM NaCl (pH 7.2), the derivatized VLPs are mixed for 2-4 h at 15° C. with a 5-fold molar excess of modified M2e with C-terminal Cys: H-Met-Ser-Leu-Leu-Thr-Glu-Val-Glu-Thr-Pro-Thr-Arg-Asn-Glu-Trp-Glu-Cys-Arg-Cys-Ser-Asp-Ser-Ser-Asp-Cys (SEQ ID NO: 20) under argon in dark to allow chemical cross-linking between the maleimide groups on the VLPs and the C-terminal Cys thiol group on the modified M2e. Uncoupled M2e peptide is then removed by extensive dialysis against PBS. The resulting VLP-M2e conjugates are then diluted with PBS for analysis and immunization.

### Example 4

### Synthetic Nanocarriers with Covalently Coupled M2e Peptide

A peptide obtained or derived from M2e, and having C-terminal alkyne linker (propargyl amide), was prepared by solid-phase peptide synthesis (Bachem Inc. Lot No. B06544, MW 2651, as acetate salt): H-Met-Ser-Leu-Leu-Thr-Glu-Val-Glu-Thr-Pro-Thr-Arg-Asn-Glu-Trp-Glu-Cys-Arg-Cys-Ser-Asp-Gly-Gly-propargyl amide (SEQ ID NO: 21).

Synthetic nanocarriers (NCs) were made by Water-oil-Water (WOW) double-emulsion evaporation process consisting of 25% wt of PLA-PEG-N3 (prepared by ring opening polymerization of HO-PEG-N3 with dl-lactide catalyzed by Sn(Oct)₂), 50% wt of PLGA-R848 (a conjugate of poly-lactide-co-glycolide and resiquimod), 25% wt of polylactic acid (100□L2A) and ova peptide (as T-cell antigen, ovalbumin peptide residues 323-339, sequence: H-Ile-Ser-Gln-Ala-Val-His-Ala-Ala-His-Ala-Glu-Ile-Asn-Glu-Ala-Gly-Arg-NH2 (SEQ ID NO: 22), acetate salt, Lot# B06395, prepared by Bachem Biosciences, Inc.). The loading of R848 and ova peptide was 2.9% wt of NCs and 0.9% wt of NCs, respectively.

Peptide were coupled to the NCs by CuAAC click chemistry as follows: The peptide (8 mg) was dissolved in 0.8 mL of PBS (pH 7.4) and mixed with 24 mg of NCs in 3 mL PBS at rt under argon in dark. To the NCs and the peptide suspension was added a solution of CuSO₄ (0.09 mL, 50 mM in water), followed by sodium ascorbate (0.09 mL, 250 mM in water). The resulting light yellow suspension was mixed gently at room temperature for 16 h. The NC-peptide suspension was then pellet-washed with PBS (2×8 mL) and re-suspended in 3 mL PBS for further analysis and bioassays.

In the same manner, NC-M2e peptide conjugates without encapsulated ova peptide were prepared and used as control in bioassays.

### Example 5

### Synthetic Nanocarriers with Covalently Coupled M2e Peptides (In Vivo Experiments)

Synthetic nanocarriers were produced according to Example 4 above (NC-M2e).

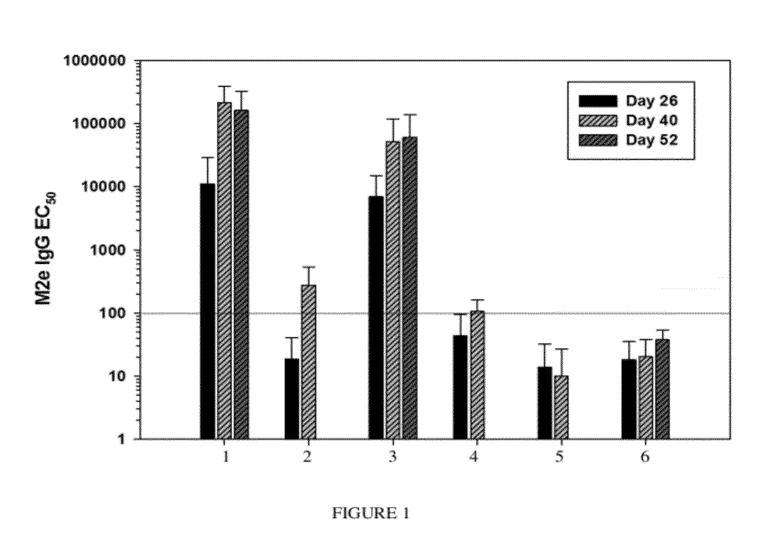

Anti-M2e antibody titers after NC-M2e vaccination (five naïve C57BL/6 female mice per group, 3 immunizations with 14-day intervals) are shown in FIG. 1. Groups 1 and 3 were immunized with 100 μg of NC-M2e (R848+ ovalbumin memory peptide); groups 2 and 4 were immunized with 100 μg of NC-M2e (R848, no ovalbumin memory peptide); group 5 was immunized with M2e peptide only (20 μg); group 6 was immunized with M2e peptide (20 μg) with alum adjuvant (1:1). Anti-M2e antibody was measured in standard ELISA at times shown (day 0=initial immunization).

Inoculation of animals with 100 μg of synthetic nanocarriers with peptides obtained or derived from M2e made according to Example 4 above resulted in efficient induction of M2e-specific antibodies, as shown in FIG. 1. Titers of anti-M2e antibodies were significantly (more than 1000-fold) higher than those generated by immunization with M2e alone or M2e admixed with a standard commercial alum adjuvant (1:1, w/w, Thermo Scientific) and were maintained for several weeks (FIG. 1).

### Example 6

### Preparation of Exemplary Nanocarriers

Example nanocarriers were produced in a two-step process. First a base nanocarrier was formed with a reactive linkage site on the surface. Second the antigen was coupled to the linkage site on the nanocarrier surface by covalent reaction chemistry. The linkage chemistry in this example was the reaction of a terminal azide group on the nanocarrier with a terminal propargyl group on the peptide antigen.

Materials: Ovalbumin peptide 323-339 amide acetate salt, was purchased from Bachem Americas Inc. (3132 Kashiwa Street, Torrance Calif. 90505. Product code 4065609.)

PLGA-R848, poly-D/L-lactide-co-glycolide, 4-amino-2-(ethoxymethyl)-α,α-dimethyl-1H-imidazo[4,5-c]quinoline-1-ethanol amide of approximately 7,400 Da made from PLGA of 3:1 lactide to glycolide ratio and having approximately 11% w/w conjugated resiquimod content was synthesized.

PLA-PEG(2K)-C6-Azide, block co-polymer consisting of a poly-D/L-lactide (PLA) block of approximately 23000 Da and a polyethylene glycol (PEG) block of approximately 2000 Da Mn that is terminated by an azide functional group at the end of a linear six-carbon alkane, was synthesized from commercial starting materials by coupling HO-PEG-COOH to 6-azidohexan-1-amine, and then generating the PLA block by ring-opening polymerization of dl-lactide with the HO-PEG-C6-Azide.

PLA-PEG(5K)-C6-Azide, block co-polymer consisting of a poly-D/L-lactide (PLA) block of approximately 22000 Da and a polyethylene glycol (PEG) block of approximately 5000 Da Mn that is terminated by an azide functional group at the end of a linear six-carbon alkane, was synthesized from commercial starting materials by coupling HO-PEG-COOH to 6-azidohexan-1-amine, and then generating the PLA block by ring-opening polymerization of dl-lactide with the HO-PEG-C6-Azide.

PLA with an inherent viscosity of 0.21 dL/g was purchased from SurModics Pharmaceuticals (756 Tom Martin Drive, Birmingham, Ala. 35211. Product Code 100 □L 2A.)

Polyvinyl alcohol (Mw=11,000-31,000,87-89% hydrolyzed) was purchased from J. T. Baker (Part Number U232-08).

Step 1: Base Nanocarrier Production: Solutions were prepared as follows: Solution 1: Ovalbumin peptide 323-339 at 20 mg/mL was prepared in 0.13N HCl at room temperature. Solution 2: PLGA-R848 at 50 mg/mL, PLA-PEG(2k)-C6-Azide or PLA-PEG(5k)-C6-Azide at 25 mg/mL, and PLA at 25 mg/mL in dichloromethane was prepared by dissolving each polymer separately in dichloromethane at 100 mg/mL then combining 2 parts PLGA-R848 solution to 1 part PLA-PEG-C6-Azide solution to 1 part PLA solution. Solution 3: Polyvinyl alcohol @ 50 mg/mL in 100 mM in 100 mM phosphate buffer, pH 8. Solution 4: 70 mM phosphate buffer, pH 8.

A primary (W1/O) emulsion was first created using Solution 1 & Solution 2. Solution 1 (0.2 mL) and Solution 2 (1.0 mL) were combined in a small glass pressure tube, mixed by repeat pipetting to form a coarse dispersion, and sonicated at 50% amplitude for 40 seconds using a Branson Digital Sonifier 250.

A secondary (W1/O/W2) emulsion was then formed by adding Solution 3 (2.0 mL) to the primary emulsion, vortexing to create a coarse dispersion, and then sonicating at 30% amplitude for 40 to 60 seconds using the Branson Digital Sonifier 250.

The secondary emulsion was added to an open 50 mL beaker containing 70 mM phosphate buffer solution (30 mL) and stirred at room temperature for 2 hours to allow the dichloromethane to evaporate and the nanocarriers to form in suspension. A portion of the suspended nanocarriers was washed by transferring the nanocarrier suspension to a centrifuge tube, spinning at 21,000 rcf for at least 45 minutes, removing the supernatant, and re-suspending the pellet in phosphate buffered saline. This washing procedure was repeated and then the pellet was re-suspended in phosphate buffered saline to achieve a nanocarrier suspension having a nominal concentration of 10 mg/mL on a polymer basis. The nanocarrier suspensions were stored frozen at −20 C until use.

TABLE 2
Base Nanocarrier Characterization:

| | Effective Diameter | TLR Agonist, | T-cell agonist, |
|---|---|---|---|
| Azide polymer | by DLS (nm) | % w/w | % w/w |
| PLA-PEG(2k)- | 234 | R848, 4.8 | Ova peptide |
| C6-Azide | | | 323-339, 2.1 |
| PLA-PEG(5k)- | 220 | R848, 5.1 | Ova peptide |
| C6-Azide | | | 323-339, 1.4 |

Step 2: Production of antigen-loaded nanocarriers: M2e-related peptide with a propargyl functional group at the C-terminal end was purchased as a custom product from CS Bio (Menlo Park, Calif.).

TABLE 3

| Peptide | Sequence |
|---|---|
| M2e | H-Met-Ser-Leu-Leu-Thr-Glu-Val-Glu-Thr-Pro-Thr-Arg-Asn-Glu-Trp-Glu-Cys- |
| | Arg-Cys-Ser-Asp-Gly-Gly-NH-CH2-CCH |
| | (SEQ ID NO: 23) |

To couple the peptide to the nanocarrier, nanocarriers were first concentrated in phosphate-buffered saline (PBS) to approximately 18 mg/mL. Next the peptide was dissolved to make a clear 2 mM solution in PBS. The peptide and nanocarrier solutions were then combined and de-gassed with argon. 100 mM CuSO₄ and 200 mM tris(hydroxypropyltriazolyl)methylamine (THPTA) were then pre-mixed and added to the nanocarrier suspension, followed by sodium ascorbate (200 mM) to achieve final concentrations of 5, 10, and 20 mM, respectively. The mixture was stirred at room temperature for 1 hour, refrigerated for 17 hours, and again at room temperature for 30 minutes. The suspension was then diluted to 5 mL in PBS, pelleted to remove the supernatant, resuspended in 10 mL PBS, and re-pelleted. The nanocarriers were resuspended a final time at 5 mg/mL in sterile PBS and stored refrigerated until use. In some exemplary nanocarriers, the PEG linker length was 2000 Da. In some exemplary nanocarriers, the PEG linker length was 5000 Da.

### Preparation of Nanocarriers for NC-Nic-OVA Conjugate:

Materials: PLGA-R848, poly-D/L-lactide-co-glycolide, 4-amino-2-(ethoxymethyl)-α,α-dimethyl-1H-imidazo[4,5-c]quinoline-1-ethanol amide of approximately 7,000 Da made from PLGA of 3:1 lactide to glycolide ratio and having approximately 8.5% w/w conjugated resiquimod content was custom manufactured at Princeton Global Synthesis (300 George Patterson Drive #206, Bristol, Pa. 19007.)

PLA-PEG-Nicotine (S-642), poly-D/L lactide-block-poly(ethylene glycol)-(±)-trans-3′-hydroxymethylnicotine ether with PEG block of approximately 5,000 Da and PLA block of approximately 21,000 Da was custom manufactured at Princeton Global Synthesis (300 George Patterson Drive #206, Bristol, Pa. 19007.)

PLA-PEG-Maleimide, block co-polymer consisting of a poly-D/L-lactide (PLA) block of approximately 22000 Da and a polyethylene glycol (PEG) block of approximately 2900 Da that is terminated by a maleimide functional group, was synthesized from commercial starting materials by generating the PLA block by ring-opening polymerization of dl-lactide with HO-PEG-Maleimide with dl-lactide.

Polyvinyl alcohol PhEur, USP (85-89% hydrolyzed, viscosity of 3.4-4.6 mPa·s) was purchased from EMD Chemicals Inc. (480 South Democrat Road Gibbstown, N.J. 08027. Part Number 4-88).

Method: Solutions were prepared as follows: Solution 1: 0.13N HCl in purified water; Solution 2: PLGA-R848 @ 50 mg/mL, PLA-PEG-Nicotine @ 25 mg/mL, and PLA-PEG-Maleimide @ 25 mg/mL in dichloromethane was prepared by dissolving each polymer separately in dichloromethane at 100 mg/mL then combining 2 parts PLGA-R848 solution to 1 part each PLA-PEG-Nicotine solution and PLA-PEG-Maleimide solution; Solution 3: Polyvinyl alcohol @ 50 mg/mL in 100 mM in 100 mM phosphate buffer, pH 8; Solution 4: 70 mM phosphate buffer, pH 8.

A primary (W1/O) emulsion was first created using Solution 1 & Solution 2. Solution 1 (0.2 mL) and Solution 2 (1.0 mL) were combined in a small glass pressure tube and sonicated at 50% amplitude for 40 seconds using a Branson Digital Sonifier 250.

A secondary (W1/O/W2) emulsion was then formed by adding Solution 3 (2.0 mL) to the primary emulsion, vortexing to create a course dispersion, and then sonicating at 30% amplitude for 40 seconds using the Branson Digital Sonifier 250.

The secondary emulsion was added to an open 50 mL beaker containing 70 mM phosphate buffer solution (30 mL) and stirred at room temperature for 2 hours to allow the dichloromethane to evaporate and the nanocarriers to form in suspension. A portion of the suspended nanocarriers was washed by transferring the nanocarrier suspension to a centrifuge tube, spinning at 21,000 rcf for 45 minutes, removing the supernatant, and re-suspending the pellet in phosphate buffered saline. This washing procedure was repeated and then the pellet was re-suspended in phosphate buffered saline to achieve a nanocarrier suspension having a nominal concentration of 10 mg/mL on a polymer basis. The nanocarrier suspension was stored frozen at −20 C until further use.

TABLE 4
Nanocarrier characterization

| Effective Diameter (nm) | TLR Agonist, % w/w | T-cell agonist, % w/w |
|---|---|---|
| 215 | R848, 4.2 | None |

### Preparation of NC-Nic-OVA Conjugate

Materials:
(1) NC with PEG-Nicotine and PEG-MAL on the surface, prepared as above; 6.5 mg/mL suspension in PBS buffer.
(2) OVA protein (Ovalbumin from egg white): Worthington, Lot# POK12101, MW: 46000.
(3) Traut's reagent (2-iminothiolane.HCl): MP Biomedical, Lot# 8830KA, MW: 137.6
(4) pH 8 buffer (sodium phosphate, 20 mM with 0.5 mM EDTA)
(5) pH 7 1× PBS buffer

Procedure: OVA protein (10 mg) was dissolved in 1 mL pH 8 buffer. A freshly made solution of Traut's reagent in pH 8 buffer (0.25 mL, 2 mg/mL) was added to the OVA protein solution. The resulting solution was stirred under argon in the dark for 1.5 h. The solution was diafiltered with MWCO 3K diafilter tube and washed with pH 8 buffer twice. The resulting modified OVA with thiol group were dissolved in 1 mL pH 8 buffer under argon. The NC suspension (3 mL, 6.5 mg/mL) was centrifuged to remove the supernatant. The modified OVA solution was then mixed with the NC pellets. The resulting suspension was stirred at rt under argon in the dark for 12 h. The NC suspension was then diluted to 10 mL with pH 7 PBS and centrifuged. The resulting NC was pellet washed with 2×10 mL pH 7 PBS. The NC-Nic-OVA conjugates were then resuspended in pH 7 PBS (ca. 6 mg/mL, 3 mL) stored at 4 C for further testing.

Preparation of Nanocarriers for NC-L2, NC-M2e, or NC-M2e-L2

Materials: Ovalbumin peptide 323-339 amide acetate salt, was purchased from Bachem Americas Inc. (3132 Kashiwa Street, Torrance Calif. 90505. Product code 4065609.)

PLGA-R848, poly-D/L-lactide-co-glycolide, 4-amino-2-(ethoxymethyl)-α,α-dimethyl-1H-imidazo[4,5-c]quinoline-1-ethanol amide of approximately 7,000 Da made from PLGA of 3:1 lactide to glycolide ratio and having approximately 8.5% w/w conjugated resiquimod content was custom manufactured at Princeton Global Synthesis (300 George Patterson Drive #206, Bristol, Pa. 19007.)

PLA-PEG-C6-N₃, block co-polymer consisting of a poly-D/L-lactide (PLA) block of approximately 23000 Da and a polyethylene glycol (PEG) block of approximately 2000 Da that is terminated by an amide-conjugated C₆H₁₂ linker to an azide, was synthesized by conjugating HO-PEG-COOH to an amino-C₆H₁₂-azide and then generating the PLA block by ring-opening polymerization of the resulting HO-PEG-C6-N3 with dl-lactide.

Polyvinyl alcohol PhEur, USP (85-89% hydrolyzed, viscosity of 3.4-4.6 mPa.$) was purchased from EMD Chemicals Inc. (480 South Democrat Road Gibbstown, N.J. 08027. Part Number 4-88).

Method: Solutions were prepared as follows: Solution 1: Ovalbumin peptide 323-339 @ 20 mg/mL was prepared in phosphate buffered saline at room temperature. Solution 2: PLGA-R848 @ 50 mg/mL and PLA-PEG-C6-N₃ @ 50 mg/mL in dichloromethane was prepared by dissolving each separately at 100 mg/mL in dichloromethane then combining in equal parts by volume. Solution 3: Polyvinyl alcohol @ 50 mg/mL in 100 mM in 100 mM phosphate buffer, pH 8. Solution 4: 70 mM phosphate buffer, pH 8.

A primary (W1/O) emulsion was first created using Solution 1 & Solution 2. Solution 1 (0.2 mL) and Solution 2 (1.0 mL) were combined in a small glass pressure tube and sonicated at 50% amplitude for 40 seconds using a Branson Digital Sonifier 250.

A secondary (W1/O/W2) emulsion was then formed by adding Solution 3 (2.0 mL) to the primary emulsion, vortexing to create a course dispersion, and then sonicating at 30% amplitude for 40 seconds using the Branson Digital Sonifier 250.

The secondary emulsion was added to an open 50 mL beaker containing 70 mM phosphate buffer solution (30 mL) and stirred at room temperature for 2 hours to allow the dichloromethane to evaporate and the nanocarriers to form in suspension. A portion of the suspended nanocarriers was washed by transferring the nanocarrier suspension to a centrifuge tube, spinning at 21,000 rcf for 45 minutes, removing the supernatant, and re-suspending the pellet in phosphate buffered saline. This washing procedure was repeated and then the pellet was re-suspended in phosphate buffered saline to achieve a nanocarrier suspension having a nominal concentration of 10 mg/mL on a polymer basis.

Two identical batches were created and then combined to form a single homogenous suspension at which was stored frozen at −20 C until further use.

TABLE 5
Azide-functionalized nanocarrier characterization

| Effective Diameter (nm) | TLR Agonist, % w/w | Antigen, % w/w |
|---|---|---|
| 209 | R848, 4.2 | Ova 323-339 peptide, 2.4 |

### Preparation of NP-L2 Conjugates

Materials:
(1) Nanoparticles with surface PEG-C6-N3 containing PLGA-R848 and Ova-peptide, prepared as above, 7 mg/mL suspension in PBS.
(2) HPV16 L2 peptide modified with an alkyne linker attached to C-terminal Lys amino group; Bachem Americas, Inc, Lot B06055, MW 2595, TFA salt; Sequence: H-Ala-Thr-Gln-Leu-Tyr-Lys-Thr-Cys-Lys-Gln-Ala-Gly-Thr-Cys-Pro-Pro-Asp-Ile-Ile-Pro-Lys-Val-Lys(5-hexynoyl)-NH2(with Cys-Cys disulfide bond, SEQ ID NO: 24)
(3) Catalysts: CuSO4, 100 mM in DI water; THPTA ligand, 200 mM in DI water; sodium ascorbate, 200 mM in DI water freshly prepared.
(4) pH 7.4 PBS buffer

Procedures: The NP suspension (7 mg/mL, 4 mL) was concentrated to ca. 1 mL in volume by centrifugation. A solution of L2 peptide (20 mg) in 2 mL PBS buffer was added. A pre-mixed solution of 0.2 mL of CuSO4 (100 mM) and 0.2 mL of THPTA ligand (200 mM) was added, followed by 0.4 mL of sodium ascorbate (200 mM). The resulting light yellow suspension was stirred in dark at ambient room temperature for 18 h. The suspension was then diluted with PBS buffer to 10 mL and centrifuged to remove the supernatant. The NP-L2 conjugates were further pellet washed twice with 10 mL PBS buffer and resuspended in pH 7.4 buffer at final concentration of ca. 6 mg/mL (ca. 4 mL) and stored at 4 C for further testing.

### Preparation of NP-M2e Conjugates

Materials:
(1) Nanoparticles with surface PEG-C6-N3 containing PLGA-R848 and Ova-peptide, prepared as above, 7 mg/mL suspension in PBS.
(2) M2e peptide modified with an alkyne linker attached to C-terminal Gly; CS Bio Co, Catalog No. CS4956, Lot: H308, MW 2650, TFA salt; Sequence: H-Met-Ser-Leu-Leu-Thr-Glu-Val-Glu-Thr-Pro-Thr-Arg-Asn-Glu-Trp-Glu-Cys-Arg-Cys-Ser-Asp-Gly-Gly-NHCH2CCH (SEQ ID NO: 25). In some embodiments, In embodiments, sequence: H-Met-Ser-Leu-Leu-Thr-Glu-Val-Glu-Thr-Pro-Ile-Arg-Asn-Glu-Trp-Glu-Cys-Arg-Cys-Ser-Asp-Gly-Gly-NHCH2CCH (SEQ ID NO: 26) could instead be used.
(3) Catalysts: CuSO4, 100 mM in DI water; THPTA ligand, 200 mM in DI water; sodium ascorbate, 200 mM in DI water freshly prepared
(4) pH 7.4 PBS buffer

Procedures: The NP suspension (7 mg/mL, 4 mL) was concentrated to ca. 1 mL in volume by centrifugation. A solution of M2e peptide (20 mg) in 2 mL PBS buffer was added. A pre-mixed solution of 0.2 mL of CuSO4 (100 mM) and 0.2 mL of THPTA ligand (200 mM) was added, followed by 0.4 mL of sodium ascorbate (200 mM). The resulting light yellow suspension was stirred in dark at ambient room temperature for 18 h. The suspension was then diluted with PBS buffer to 10 mL and centrifuged to remove the supernatant. The NP-M2e conjugates were further pellet washed twice with 10 mL PBS buffer and resuspended in pH 7.4 buffer at final concentration of ca. 6 mg/mL (ca. 4 mL) and stored at 4 C for further testing.

### Example 7

### Synthetic Nanocarriers with Covalently Coupled M2e Peptides Using Two Different Types of PEG (In Vivo Experiments)

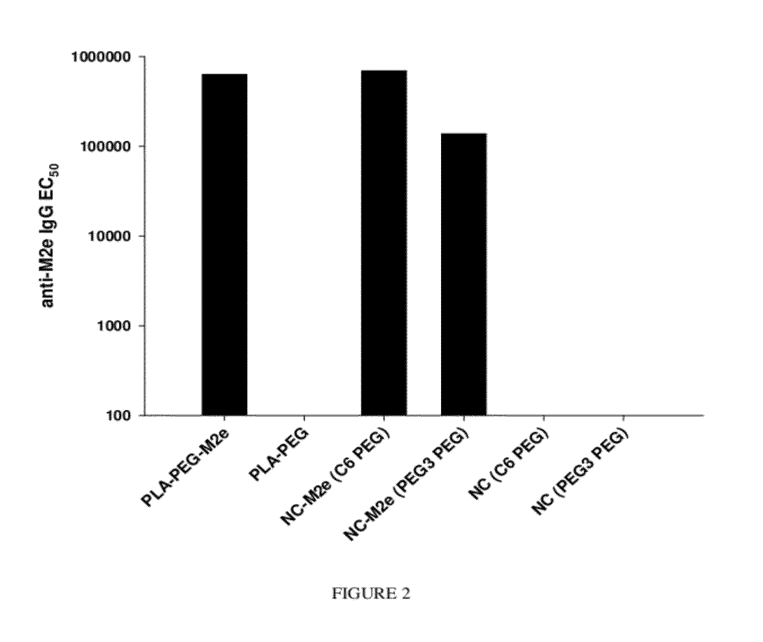

Synthetic nanocarriers were prepared according to Examples above (NC-M2e). Prior to use for immunization of mice, the presence of M2e on nanocarriers was confirmed by ELISA using a monoclonal anti-M2e peptide antibody (AbCam). Both nanocarriers contained M2e peptide that was recognized by the anti-M2 antibody (FIG. 2). The antibody reacted with the positive control (PLA-PEG-M2e) and did not react to the negative controls (PLA-PEG and no peptide nanocarriers). Two types of NC-Me2 were characterized: NC-M2e (C6 PEG), and NC-M2e (PEG3 PEG).

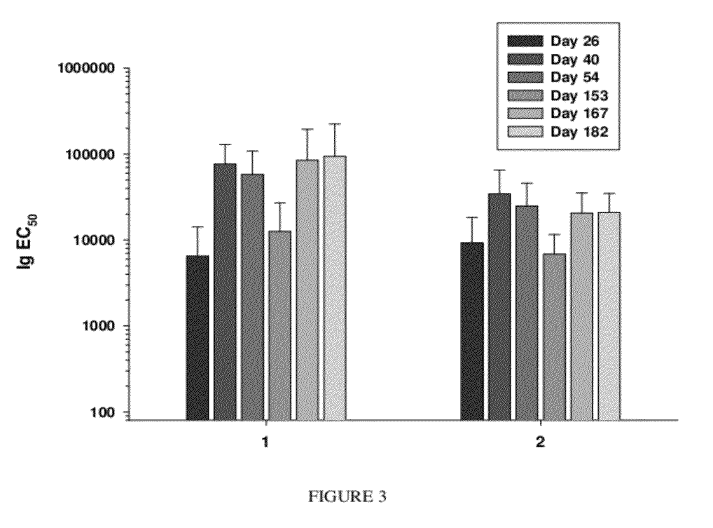

C57BL/6 mice were vaccinated using the synthetic nanocarriers (s.c., hind limbs, 60 μL total inoculation volume, 3 times with a 3-week interval and 1 time at day 155). Group 1: immunized with 100 μg of NC-M2e (C6 PEG), group 2: immunized with 100 μg of NC-M2e (PEG3 PEG). Mice were bled at days 26, 40, 54, 153, 167, and 182 and anti-M2e peptide antibody titers were determined by a standard ELISA against M2e peptide. Results are shown in FIG. 3.

Titers of anti-M2e antibodies generated by mice immunized with NC-M2e (C6 PEG) were not significantly different from those generated by mice immunized with NC-M2e (PEG3 PEG) (FIG. 3).

### Example 8

### In Vivo Testing of Synthetic Nanocarriers with Covalently Coupled M2e Peptide in the Presence of Nanocarriers with Covalently Coupled Proteins or Peptides, H5N1 Hemagglutinin Protein, or H1N1 Inactivated Virus (In Vivo Experiments)

Synthetic nanocarriers were prepared according to Examples above:

1 NP-M2e+NP-L2

2 NP-M2e+NP-L2+NP-Nic-OVA

3 NP-M2e+HA5 protein (H5N1 HA protein (Protein Sciences))

4 NP-M2e+HA5 protein+Alum (imject Alum (Pierce))

5 NP-M2e+H1N1 virus (inactivated influenza H1N1 virus (ProSpec))+Alum

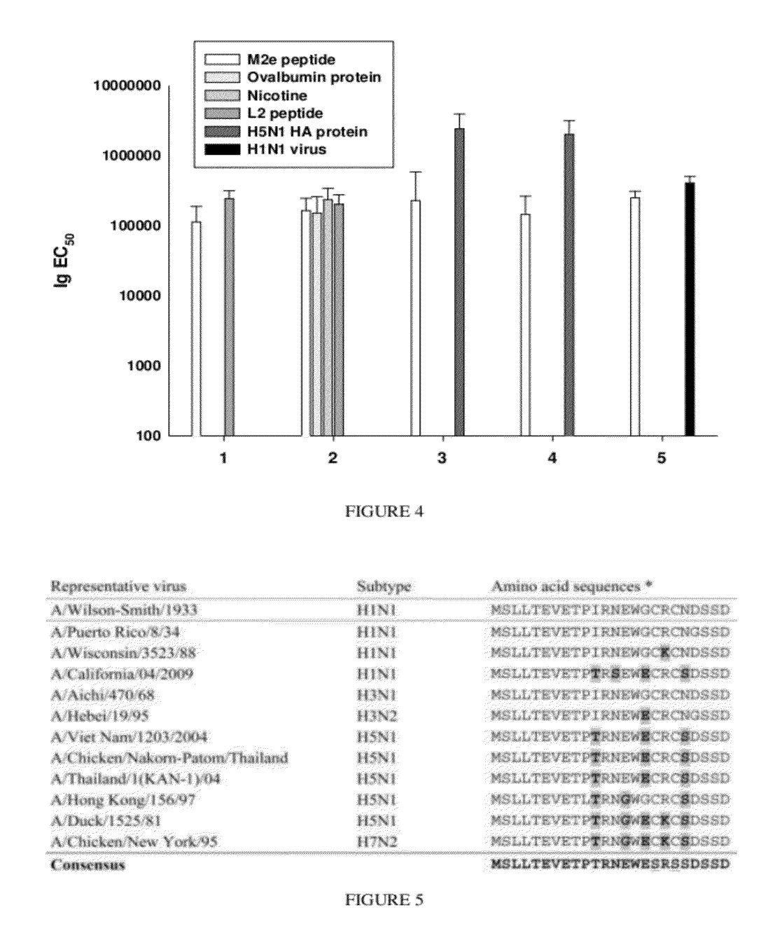

C57BL/6 mice were vaccinated using the synthetic nanocarriers (s.c., hind limbs, 60 μL total inoculation volume, 2 times with a 3-week interval). Group 1: immunized with 100 μg of nanoparticle-M2e peptide conjugates (NC-M2e) and 100 μg of nanoparticle-L2 peptide conjugates (NC-L2), group 2: immunized with 100 μg of NC-M2e, 100 μg of NC-L2, and 100 μg of nanoparticle-nicotine and ovalbumin protein conjugates (NC-Nic-OVA), group 3: immunized with 100 μg of NC-M2e and 10 μg of H5N1 hemagglutinin protein (HA5 protein); group 4: immunized with 100 μg NC-M2e and 10 μg HA5 protein plus alum (1:1); group 5: immunized with 100 μg NC-M2e and 10 μg of inactivated H1N1 influenza virus. Mice were bled at day 33 and anti-HA5 protein, anti-OVA, anti-M2e peptide, anti-nicotine, anti-H1N1 virus, and anti-L2 peptide antibody titers were determined by a standard ELISA against H5N1 HA protein, OVA protein, M2e peptide, nicotine, H1N1 inactivated virus, or L2 peptide. Results are shown in FIG. 4.

Titers of anti-M2e antibodies generated by mice immunized with both NC-M2e and NC-L2 were comparable to those generated by mice immunized with NC-M2e alone (FIGS. 3 and 4). These mice also generated antibodies to HPV L2 peptide (FIG. 4). Titers of anti-M2e antibodies generated by mice immunized with three nanocarriers (NC-M2e, NC-L2, and NC-Nic-OVA) were comparable to those generated by mice immunized with NC-M2e alone (FIGS. 3 and 4). In addition, they generated antibodies to L2 peptide, nicotine, and ovalbumin (FIG. 4). Mice immunized with both NC-M2e and influenza H5N1 HA protein (±alum) or NC-M2e and inactivated H1N1 influenza virus generated antibodies to M2e peptide at levels comparable to those generated by mice immunized with NC-M2e alone (FIGS. 3 and 4). In addition, these mice generated antibodies to H5N1 HA protein or H1N1 inactivated influenza virus (FIG. 4).

### REFERENCES

Beerli R. R., Bauer M., Schmitz N., et al. Prophylactic and therapeutic activity of fully human monoclonal antibodies directed against influenza A M2 protein. Virol. J. 2009; 6:224.
Black, M., Trent A., Tirrel M., and Olive, C. Advances in the design and delivery of peptide subunit vaccines with a focus on Toll-like receptor agonists. Expert Rev. Vaccines 2010; 9:157-173.
Black R. A., Rota P. A., Gorodkova N., et al. Production of the M2 protein of influenza A virus in insect cells is enhanced in the presence of amantadine. J. Gen. Virol. 1993; 74:1673-1677.
Caton, A. J., Brownlee, G. G., Yewdell, J. M. and Gerhard, W. The antigenic structure of the influenza virus A/PR/8/34 hemagglutinin (H1 subtype). Cell. 1982; 31:417-427.
Cross K. J., Langley W. A., Russell R. J., et al. Composition and functions of the influenza fusion peptide. Protein Pept. Lett. 2009; 16:766-778.
De Filette M., Fiers W., Martens W., et al. Improved design and intranasal delivery of an M2e-based human influenza A vaccine. Vaccine. 2006; 24:6597-6601.
De Filette M., Martens W., Roose K., et al. An influenza A vaccine based on tetrameric ectodomain of matrix protein 2. J. Biol. Chem. 2008a; 283:11382-11387.
De Filette M., Martens W., Smet A., et al. Universal influenza A M2e-HBc vaccine protects against disease even in the presence of pre-existing anti-HBc antibodies. Vaccine. 2008b; 26:6503-6507.
De Filette M., Min Jou W., Birkett A., et al. Universal influenza A vaccine: optimization of M2-based constructs. Virology. 2005; 337:149-161.
Denis J., Acosta-Ramirez E., Zhao Y., et al. Development of a universal influenza A vaccine based on the M2e peptide fused to the papaya mosaic virus (PapMV) vaccine platform. Vaccine. 2008; 26:3395-3403.
Ellebedy A. H., Webby R. J. Influenza vaccines. Vaccine. 2009; 27 Suppl. 4:D65-8.
Ernst W. A., Kim H. J., Tumpey T. M., et al. Protection against H1, H5, H6 and H9 influenza A infection with liposomal matrix 2 epitope vaccines. Vaccine. 2006; 24:5158-5168.
Fan J., Liang X., Horton M. S., et al. Preclinical study of influenza virus A M2 peptide conjugate vaccines in mice, ferrets, and rhesus monkeys. Vaccine. 2004; 22:2993-3003.
Huleatt J. W., Nakaar V., Desai P., et al. Potent immunogenicity and efficacy of a universal influenza vaccine candidate comprising a recombinant fusion protein linking influenza M2e to the TLR5 ligand flagellin. Vaccine. 2008; 26:201-214.
Jegerlehner A., Schmitz N., Storni T., Bachmann M. F. Influenza A vaccine based on the extracellular domain of M2: weak protection mediated via antibody-dependent NK cell activity. J. Immunol. 2004; 172:5598-5605.
Jimenez G. S., Planchon R., Wei Q., et al. Vaxfectin-formulated influenza DNA vaccines encoding NP and M2 viral proteins protect mice against lethal viral challenge. Hum. Vaccin. 2007; 3:157-164.
Mozdzanowska K., Feng J., Eid M., et al. Induction of influenza type A virus-specific resistance by immunization of mice with a synthetic multiple antigenic peptide vaccine that contains ectodomains of matrix protein 2. Vaccine. 2003; 21:2616-2626.
Mozdzanowska K., Zharikova D., Cudic M., et al. Roles of adjuvant and route of vaccination in antibody response and protection engendered by a synthetic matrix protein 2-based influenza A virus vaccine in the mouse. Virol. J. 2007; 4:118.
Neirynck S., Deroo T., Saelens X., et al. A universal influenza A vaccine based on the extracellular domain of the M2 protein. Nat. Med. 1999; 5:1157-1163.
Purcell, A. W., Zeng, W., Mifsud, N. A., et al. Dissecting the Role of Peptides in the Immune Response Theory, Practice and the Application to Vaccine Design. J. Peptide Sci. 2003; 9: 255-281.
Roose K., Fiers W., Saelens X. Pandemic preparedness: toward a universal influenza vaccine. Drug News Perspect. 2009; 22:80-92.
Schotsaert M., De Filette M., Fiers W., Saelens X. Universal M2 ectodomain-based influenza A vaccines: preclinical and clinical developments. Expert Rev. Vaccines. 2009; 8:499-508.
Slepushkin V. A., Katz J. M., Black R. A., et al. Protection of mice against influenza A virus challenge by vaccination with baculovirus-expressed M2 protein. Vaccine. 1995; 13:1399-1402.
Sui J., Hwang W. C., Perez S., et al. Structural and functional bases for broad-spectrum neutralization of avian and human influenza A viruses. Nat Struct Mol. Biol. 2009; 16:265-273.
Throsby M., van den Brink E., Jongeneelen M., et al. Heterosubtypic neutralizing monoclonal antibodies cross-protective against H5N1 and H1N1 recovered from human IgM+ memory B cells. PLoS One. 2008; 3:e3942.
Tompkins S. M., Zhao Z. S., Lo C. Y., et al. Matrix protein 2 vaccination and protection against influenza viruses, including subtype H5N1. Emerg. Infect. Dis. 2007; 13:426-435.
Treanor J. J., Tierney E. L., Zebedee S. L., et al. Passively transferred monoclonal antibody to the M2 protein inhibits influenza A virus replication in mice. J. Virol. 1990; 64:1375-1377.
Tsuchiya, E., Sugawara, K., Hongo, S., Matsuzaki, Y., Muraki, Y., L1, Z.-N. and Nakamura, K. Antigenic structure of the haemagglutinin of human influenza A/H2N2 virus. Journal of General Virology 2001; 82:2475-2484.
Vu Hong, Stanislav I. Presolski, Celia Ma, M. G. Finn; Analysis and Optimization of Copper-Catalyzed Azide—Alkyne Cycloaddition for Bioconjugation”; Angewandte Chemie International Edition; 2009, 48: 9879-9883.
Wang R., Song A., Levin J., et al. Therapeutic potential of a fully human monoclonal antibody against influenza A virus M2 protein. Antiviral Res. 2008; 80:168-177.

1. A dosage form comprising synthetic nanocarriers coupled to peptides that are obtained or derived from an ectodomain region of human influenza A virus M2 protein.

2. (canceled)

3. The dosage form of claim 1, wherein the peptides comprise a peptide obtained or derived from a peptide with an amino acid sequence as set forth in any of SEQ ID NOs: 1-17, 19-21, 23, 25 and 26.

4. The dosage form of claim 1, wherein the synthetic nanocarriers are further coupled to one or more adjuvants.

5-6. (canceled)

7. The dosage form of claim 1, wherein the synthetic nanocarriers comprise lipid-based nanoparticles, polymeric nanoparticles, metallic nanoparticles, surfactant-based emulsions, dendrimers, buckyballs, nanowires, virus-like particles, peptide or protein-based particles, lipid-polymer nanoparticles, spheroidal nanoparticles, cubic nanoparticles, pyramidal nanoparticles, oblong nanoparticles, cylindrical nanoparticles, or toroidal nanoparticles, and, optionally, wherein the synthetic nanocarriers comprise poly(lactic acid)-polyethyleneglycol copolymer, poly(glycolic acid)-polyethyleneglycol copolymer, or poly(lactic-co-glycolic acid)-polyethyleneglycol copolymer.

8. (canceled)

9. The dosage form of any of claim 1, wherein the synthetic nanocarriers comprise a T-helper antigen.

10. (canceled)

11. The dosage form of claim 1, further comprising influenza antigen that is not coupled to the synthetic nanocarriers.

12. The dosage form of claim 1, wherein at least a portion of the peptides that are obtained or derived from an ectodomain region of human influenza A virus M2 protein are coupled to a surface of the synthetic nanocarriers.

13-15. (canceled)

16. The dosage form of claim 1, wherein the dosage form generates in a subject polyclonal antibodies that compete for binding to human influenza A virus M2 protein with a control antibody, wherein the control antibody is 14C2.

17. A dosage form comprising peptides that are obtained or derived from an ectodomain region of human influenza A virus M2 protein, wherein the dosage form generates in a subject polyclonal antibodies that compete for binding to human influenza A virus M2 protein with a control antibody, wherein the control antibody is 14C2.

18. (canceled)

19. The dosage form of claim 17, wherein the peptides comprise a peptide obtained or derived from a peptide with an amino acid sequence as set forth in any of SEQ ID NOs: 1-17, 19-21, 23, 25 and 26.

20. The dosage form of claim 17, wherein the dosage form further comprises one or more adjuvants.

21-22. (canceled)

23. The dosage form of claim 17, wherein the dosage form further comprises T-helper antigens.

24. The dosage form of claim 17, wherein the dosage form further comprises a carrier that boosts an immune response to the peptides when administered to a subject.

25. The dosage form of claim 24, wherein the peptides are coupled to the carrier.

26. The dosage form of claim 24, wherein the carrier comprises keyhole limpet hemocyanin, concholepas concholepas hemocyanin, bovine serum albumin, cationized BSA or ovalbumin.

27. The dosage form of claim 24, wherein the carrier comprises a synthetic nanocarrier.

28. The dosage form of claim 27, wherein the synthetic nanocarrier comprises a/an lipid-based nanoparticle, polymeric nanoparticle, metallic nanoparticle, surfactant-based emulsion, dendrimer, buckyball, nanowire, virus-like particle, peptide or protein-based particle, lipid-polymer nanoparticle, spheroidal nanoparticle, cubic nanoparticle, pyramidal nanoparticle, oblong nanoparticle, cylindrical nanoparticle, or toroidal nanoparticle, and, optionally, wherein the synthetic nanocarrier comprises poly(lactic acid)-polyethyleneglycol copolymer, poly(glycolic acid)-polyethyleneglycol copolymer, or poly(lactic-co-glycolic acid)-polyethyleneglycol copolymer.

29-31. (canceled)

32. The dosage form of claim 17, further comprising influenza antigen or, when the dosage form further comprises a carrier, influenza antigen that is not coupled to the carrier.

33. A method comprising administering the dosage form of claim 1 to a subject.

34-37. (canceled)

38. A method comprising:
providing synthetic nanocarriers; and
coupling peptides that are obtained or derived from an ectodomain region of human influenza A virus M2 protein to the synthetic nanocarriers.

39. The method of claim 38, wherein coupling comprises covalently coupling the peptides to the synthetic nanocarriers.

40. A composition, dosage form or vaccine obtained, or obtainable, by a method as defined in claim 38.

41. A process for producing a composition, dosage form or vaccine comprising the steps of:
providing synthetic nanocarriers; and
coupling peptides that are obtained or derived from an ectodomain region of human influenza A virus M2 protein to the synthetic nanocarriers.

42-47. (canceled)


SEQUENCE LISTING (26 sequences)

SEQ ID NO: 1 - protein, 24 aa, ARTIFICIAL SEQUENCE
  synthetic polypeptide
MSLLTEVETPIRNEWGCRCNDSSD

SEQ ID NO: 2 - protein, 24 aa, ARTIFICIAL SEQUENCE
  synthetic polypeptide
MSLLTEVETPIRNEWGCRCNGSSD

SEQ ID NO: 3 - protein, 24 aa, ARTIFICIAL SEQUENCE
  synthetic polypeptide
MSLLTEVETPIRNEWGCKCNDSSD

SEQ ID NO: 4 - protein, 24 aa, ARTIFICIAL SEQUENCE
  synthetic polypeptide
MSLLTEVETPTRSEWECRCSDSSD

SEQ ID NO: 5 - protein, 24 aa, ARTIFICIAL SEQUENCE
  synthetic polypeptide
MSLLTEVETPIRNEWGCRCNDSSD

SEQ ID NO: 6 - protein, 24 aa, ARTIFICIAL SEQUENCE
  synthetic polypeptide
MSLLTEVETPIRNEWECRCNGSSD

SEQ ID NO: 7 - protein, 24 aa, ARTIFICIAL SEQUENCE
  synthetic polypeptide
MSLLTEVETPTRNEWECRCSDSSD

SEQ ID NO: 8 - protein, 24 aa, ARTIFICIAL SEQUENCE
  synthetic polypeptide
MSLLTEVETPTRNEWECRCSDSSD

SEQ ID NO: 9 - protein, 24 aa, ARTIFICIAL SEQUENCE
  synthetic polypeptide
MSLLTEVETPTRNEWECRCSDSSD

SEQ ID NO: 10 - protein, 24 aa, ARTIFICIAL SEQUENCE
  synthetic polypeptide
MSLLTEVETPTRNGWGCRCSDSSD

SEQ ID NO: 11 - protein, 24 aa, ARTIFICIAL SEQUENCE
  synthetic polypeptide
MSLLTEVETPTRNGWECKCSDSSD

SEQ ID NO: 12 - protein, 24 aa, ARTIFICIAL SEQUENCE
  synthetic polypeptide
MSLLTEVETPTRNGWECKCSDSSD

SEQ ID NO: 13 - protein, 24 aa, ARTIFICIAL SEQUENCE
  synthetic polypeptide
MSLLTEVETPTRNEWESRSSDSSD

SEQ ID NO: 14 - protein, 24 aa, ARTIFICIAL SEQUENCE
  synthetic polypeptide
MSLLTEVETPTRNEWECRCSDSSD

SEQ ID NO: 15 - protein, 23 aa, ARTIFICIAL SEQUENCE
  synthetic polypeptide
MSLLTEVETPTRNEWECRCSDGG

SEQ ID NO: 16 - protein, 25 aa, ARTIFICIAL SEQUENCE
  synthetic polypeptide
MSLLTEVETPTRNEWESRSSDSSDC

SEQ ID NO: 17 - protein, 26 aa, ARTIFICIAL SEQUENCE
  synthetic polypeptide
MSLLTEVETPIRNEWGCRCNDSSDXC

SEQ ID NO: 18 - protein, 17 aa, ARTIFICIAL SEQUENCE
  synthetic polypeptide
ISQAVHAAHAEINEAGR

SEQ ID NO: 19 - protein, 25 aa, ARTIFICIAL SEQUENCE
  synthetic polypeptide
MSLLTEVETPTRNEWECRCSDSSDC

SEQ ID NO: 20 - protein, 25 aa, ARTIFICIAL SEQUENCE
  synthetic polypeptide
MSLLTEVETPTRNEWECRCSDSSDC

SEQ ID NO: 21 - protein, 23 aa, ARTIFICIAL SEQUENCE
  synthetic polypeptide
MSLLTEVETPTRNEWECRCSDGG

SEQ ID NO: 22 - protein, 17 aa, ARTIFICIAL SEQUENCE
  synthetic polypeptide
ISQAVHAAHAEINEAGR

SEQ ID NO: 23 - protein, 23 aa, ARTIFICIAL SEQUENCE
  synthetic polypeptide
MSLLTEVETPTRNEWECRCSDGG

SEQ ID NO: 24 - protein, 23 aa, ARTIFICIAL SEQUENCE
  synthetic polypeptide
ATQLYKTCKQAGTCPPDIIPKVK

SEQ ID NO: 25 - protein, 23 aa, ARTIFICIAL SEQUENCE
  synthetic polypeptide
MSLLTEVETPTRNEWECRCSDGG

SEQ ID NO: 26 - protein, 23 aa, ARTIFICIAL SEQUENCE
  synthetic polypeptide
MSLLTEVETPIRNEWECRCSDGG